(19) United States
(12) Patent Application Publication
(10) Pub. No.: US 2006/0135963 A1
(43) Pub. Date: Jun. 22, 2006

(54) Expandable gastrointestinal sheath

(76) Inventors: George F. Kick, Casa Grande, AZ (US); Jay Lenker, Laguna Beach, CA (US); Edward J. Nance, Corona, CA (US); Onnik Tchulluian, Carlsbad, CA (US); Joseph Bishop, Menifee, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/222,499

(22) Filed: Sep. 8, 2005

Related U.S. Application Data
(60) Provisional application No. 60/659,831, filed on Mar. 9, 2005.
(60) Provisional application No. 60/608,355, filed on Sep. 9, 2004.

(51) Int. Cl. A61F 11/00

(52) U.S. Cl. 606/108; 606/191

(57) ABSTRACT

Disclosed is an expandable transluminal sheath, for introduction into the body while in a first, low cross-sectional area configuration, and subsequent expansion of at least a part of the distal end of the sheath to a second, enlarged cross-sectional configuration. The sheath is configured for use in the gastrointestinal system and has utility in the performance of endoscopic retrograde cholangiopancreatography (ERCP). The distal end of the sheath is maintained in the first, low cross-sectional configuration and expanded using a radial dilatation device. In an exemplary application, the sheath is utilized to provide access for a diagnostic or therapeutic procedure such as gallstone or pancreatic stone removal.

### PRIORITY CLAIM

This application claims the benefit of U.S. Provisional Application No. 60/659,831, filed on Mar. 9, 2005, and U.S. Provisional Application No. 60/608,355, filed on Sep. 9, 2004, the entirety of these applications are hereby incorporated by reference herein.

### BACKGROUND OF THE INVENTION

1. Field of the Invention

The invention relates to medical devices and, more particularly, to methods and devices for accessing a gastrointestinal tract.

2. Description of the Related Art

A wide variety of diagnostic or therapeutic procedures involves the introduction of a device through a natural access pathway such as a body lumen or cavity. A general objective of access systems, which have been developed for this purpose, is to minimize the cross-sectional area of the access lumen, while maximizing the available space for the diagnostic or therapeutic instrumentation. These procedures are especially suited for the gastrointestinal (GI) tract of the human or other mammal, including the esophagus, stomach, duodenum, small intestine and organ outflow tracts such as the bile duct and pancreatic duct. Other applications include procedures in the bronchial and tracheal passages, and the lower GI tract including the colon and the anus.

Endoscopic retrograde cholangiopancreatography (ERCP) is an example of one type of therapeutic or diagnostic interventional procedure that relies on natural access pathways such as the esophagus, the stomach, which is a body cavity, the duodenum, the small intestine, and the common bile and pancreatic ducts. Access to the gastrointestinal tract is gained through the nose or throat into the esophagus. During the procedure, a flexible, right-angle viewing endoscope is routed into an upper part of the small intestine, called the descending duodenum, to the sphincter of hepatopancreatic ampulla, at the entrance to the bile ducts. A guidewire and catheter are inserted through the working channel of the endoscope, through the sphincter, sometimes called the papilla or sphincter of Oddi, into the bile ducts so that radiopaque dye, generally comprising barium salts, can be injected therein to facilitate fluoroscopic and X-ray evaluation of the anatomy. ERCP is also used to route graspers into the bile and pancreatic ducts for the removal of calculi. It is also used for acquisition of biopsy samples and the placement of stents, both temporary and permanent.

To perform a procedure in either the bile or pancreatic duct, an endoscope is placed into the duodenum through the esophagus, a body lumen, and the stomach, a body cavity. A guidewire, generally 0.018 to 0.038 inches in diameter but preferably 0.035 inches in diameter, is next routed, through the working channel of the endoscope and under direct visual guidance, deflected sideways, through the papilla, into the bile duct or pancreatic duct. Once guidewire control is established, a diagnostic catheter is advanced over the guidewire with the deflecting endoscope, generally a right-angle viewing endoscope, left in place. Injection of radiopaque dye allows fluoroscopic visualization of the ducts. Areas of stones or calculi show up as regions not penetrated by the dye. Calculi, largely consisting of cholesterol or, more rarely, based on calcium, are not readily visible under fluoroscopy, X-ray or computer-aided tomography (CT) so only the absence of dye can be used to see their presence using these detection systems. The calculi may be visible, however, using ultrasound or magnetic resonance imaging (MRI).

Current therapeutic techniques may involve advancing a steerable, flexible, right-angle viewing, endoscope, generally as large as or larger than 15 French, to the external aspect of the papilla. Prior to performing therapeutic procedures such as stone removal, a sphincterotomy may be performed, through the endoscope, to cut the sphincter of hepatopancreatic ampulla, to gain access to the duct so that stones can be removed therethrough. Provision is generally required to deflect instrumentation through large angles coming out of the endoscope because the common bile duct and the pancreatic duct approach the duodenum at an angle between 90 degrees and 180 degrees from the direction of catheterization. The actual entrance to the common bile duct, from which the pancreatic duct is generally, but not always, a side branch, is at approximately a 90-degree to 120-degree angle to the axis of the duodenum. Once inside the common bile duct, the duct turns again through a significant angle so that it runs nearly parallel to the long axis of the duodenum. The therapeutic devices or procedures generally involve using graspers or baskets to remove stones, or catheters to deploy stents for relief of stenosis caused by tumors, for example.

One of the issues that could arise during ERCP is the need to remove and replace instruments without causing undue patient discomfort or tissue damage, which could have long or short-term after effects. Some sort of external protective sheath or cannula would be useful in this capacity. Another potentially bothersome complication of the procedure is reflux (retrograde migration) of intestinal contents or material into the pancreas causing inflammation, known as pancreatitis, which can be quite severe. Such conditions are currently accepted by physicians but patient outcomes would be improved if a sphincterotomy were not required and if catheter or endoscope replacement could be more easily and gently accomplished with less tissue trauma. Gastroenterologists may be required to use sheaths or catheters with suboptimal central lumen size because they are the largest catheters that can be advanced to the distal end of the endoscope's generally 6 to 8-French working channel. Furthermore, stent placement would be facilitated if a larger working channel could be made available than the one found on most endoscopes used for this purpose. Both temporary plastic stents and permanent metallic stents may be delivered for this purpose. The stents may be either self-expanding, balloon expandable, or non-expandable, such as the case with ureteral stents.

Further reading related to ERCP includes Alhalel, R, and Haber, GB, Endoscopic Therapy of Pancreatic Stones, Gastrointestinal Endoscopy Clinics of North America, Vol. 5, No. 1, 1995, pp 195-215. Data regarding complications of the procedure may be found in Christensen, M, Matzen, P, Schulze, S, and Rosenberg, J, Complications of ERCP: a Prospective Study, Gastrointestinal Endoscopy, Vol. 60, No. 5, 2004, pp 721-731. Additional information regarding ERCP can be found in-patient brochures on the subject published by the American Gastroenterological Association and is available online.

### SUMMARY OF THE INVENTION

Accordingly, one embodiment of the present invention comprises an expandable transluminal access sheath for providing minimally invasive access to a gastrointestinal tract. The sheath includes an axially elongate sheath tube comprising a proximal end, a distal end, and a through lumen extending therethrough. The sheath tube further comprises a distal region that is expandable from a collapsed configuration to an expanded configuration in response to outward pressure applied therein. A hub is coupled to the proximal end of the sheath tube. An obturator extends through the hub and sheath tube. The obturator is configured to occlude the central lumen of the sheath tube during insertion of the sheath tube into the gastrointestinal tract. The obturator comprises an obturator hub that is releasably coupled to the hub of the sheath and a guidewire lumen that extends through the obturator. The obturator further comprises a balloon dilator capable of expanding the distal region of the sheath from the collapsed configuration to the expanded configuration.

Another embodiment of the present invention comprises a method of instrumenting a body lumen. In the method, an endoscope with a working channel is inserted into a patient. An exit point of the working channel is positioned beside an entrance to a branch of the body lumen. A guidewire is routed down the working channel of the endoscope and into the branch of the body. An end of the guidewire is positioned at a target location within the body lumen. The endoscope is removed from the patient leaving the guidewire in place. A sheath is inserted with a collapsed distal region and a pre-inserted dilator into the patient over the guidewire. The sheath is advanced to a treatment site within the side branch of the body lumen. The distal region of the sheath is dilated so that the distal region of the sheath is expanded. The dilator is collapsed. The dilator is removed from the sheath. The instrumentation is Inserted through the lumen of the sheath. Therapy or diagnosis is performed with the instrumentation. The sheath is removed from the patient.

Another embodiment of the invention comprises an access device for insertion into a gastrointestinal tract. The device includes means for tracking over a guidewire to a target treatment site, means for diametrically collapsing at least a distal end of the sheath, means for dilating at least a portion of the distal end of the sheath, from the proximal end of the sheath, and means for removal of the sheath from the patient body lumen or cavity.

Another embodiment of the invention comprises an expandable transluminal access sheath adapted for providing minimally invasive access to the gastrointestinal tract through a working channel of an endoscope. An axially elongate sheath tube is provided with a proximal end, a distal end, and a central through lumen. A distal region of the sheath is expandable, in response to outward pressure applied therein, to a diameter which is larger than that of a proximal region of the sheath. A hub is affixed to the proximal end of the sheath tube. The hub is adapted to facilitate the passage of instrumentation.

A need therefore remains for improved access technology, which allows a device to be transesophageally, passed through the esophagus and stomach into the small intestine with a small introduction diameter, while accommodating the introduction of relatively large diameter instruments. It would be beneficial if a gastroenterologist did not need to inventory and use a range of catheter diameters. It would be far more useful if one catheter diameter could fit the majority of patients. Ideally, the catheter would be able to enter a vessel or body lumen with a diameter of 3 to 12 French or smaller, and be able to pass instruments through a central lumen that is 14 to 20 French. The sheath would be capable of gently dilating the papilla sphincter and of permitting the exchange of instrumentation therethrough without being removed from the body. The sheath would also be maximally visible under fluoroscopy and would be relatively inexpensive to manufacture. The sheath or catheter would be kink resistant and minimize abrasion and damage to instrumentation being passed therethrough. The sheath or catheter would further minimize the potential for injury to body lumen or cavity walls or surrounding structures.

One embodiment of the present invention comprises a transluminal radially expanding access sheath. The radially expanding access sheath is used to provide selective access to the common bile duct or the pancreatic duct. In an embodiment, the sheath would have an introduction outside diameter that ranged from 3 to 12 French with a preferred range of 5 to 10 French. The diameter of the sheath would be expandable to permit instruments ranging up to 30 French to pass therethrough, with a preferred range of between 3 and 20 French. The sheath can have a working length ranging between 150-cm and 300-cm with a preferred length of 175-cm to 225-cm. The ability to pass the large traditional instruments and smaller more innovative instruments through a catheter introduced with a small outside diameter is derived from the ability to expand the distal end of the catheter to create a larger through lumen. The expandable distal end of the catheter can comprise 75% or more of the overall working length of the catheter. The proximal end of the catheter is generally larger than the distal end to provide for pushability, control, and the ability to pass large diameter instruments therethrough. In an embodiment, the sheath can be routed to its destination over or alongside one or more already placed guidewires with a diameter ranging up to 0.040 inches.

Another embodiment of the invention comprises a transluminal access system for providing minimally invasive access to gastroenterological structures. The system includes an access sheath comprising an axially elongate tubular body that defines a lumen extending from the proximal end to the distal end of the sheath. At least a portion of the distal end of the elongate tubular body is expandable from a first, smaller cross-sectional profile to a second, greater cross-sectional profile. In an embodiment, the first, smaller cross-sectional profile is created by making axially oriented folds in the sheath material. These folds may be located in only one circumferential position on the sheath, or there may be a plurality of such folds or longitudinally oriented crimps in the sheath. The folds or crimps may be made permanent or semi-permanent by heat-setting the structure, once folded. In an embodiment, a releasable jacket is carried by the access sheath to restrain at least a portion of the elongate tubular structure in the first, smaller cross-sectional profile. In another embodiment, the jacket is removed prior to inserting the sheath into the patient. In an embodiment, the elongate tubular body is sufficiently pliable to allow the passage of objects having a maximum cross-sectional size larger than an inner diameter of the elongate tubular body in the second, greater cross-sectional profile. The adaptability to objects of larger dimension is accomplished by pliability or re-shaping of the cross-section to the larger dimension in one direction accompanied by a reduction in dimension in a lateral direction. The adaptability may also be generated through the use of malleable or elastomerically deformable sheath material.

In another embodiment of the invention, a transluminal access sheath assembly for providing minimally invasive access comprises an elongate tubular member having a proximal end and a distal end and defining a working inner lumen. In this embodiment, the tubular member comprises a folded or creased sheath that can be expanded by a dilatation balloon. The dilatation balloon, if filled with fluids, preferably liquids and further preferably radiopaque liquids,. at appropriate pressure, can generate the force to expand the sheath. The dilatation balloon is removable to permit subsequent instrument.passage through the sheath. Longitudinal runners may be disposed within the sheath to serve as tracks for instrumentation, which further minimize friction while minimizing the risk of catching the instrument on the expandable plastic tubular member. Such longitudinal runners are preferably circumferentially affixed within the sheath so as not to shift out of alignment. In yet another embodiment, the longitudinal runners may be replaced by longitudinally oriented ridges and valleys, termed flutes. The flutes, or runners, can be oriented along the longitudinal axis of the sheath, or they can be oriented in a spiral, or rifled, fashion.

In the embodiments describe above, the proximal end of the access assembly, apparatus, or device is preferably fabricated as a structure that is flexible, resistant to kinking, and further retains both column strength and torqueability. Such structures include tubes fabricated with coils or braided reinforcements and preferably comprise inner walls that prevent the reinforcing structures from protruding, poking through, or becoming exposed to the inner lumen of the access apparatus. Such proximal end configurations may be single lumen, or multi-lumen designs, with a main lumen suitable for instrument, guidewire, endoscope, or obturator passage and additional lumens being suitable for control and operational functions such as balloon inflation. Such proximal tube assemblies can be affixed to the proximal end of the distal expandable segments described heretofore. In an embodiment, the proximal end of the catheter includes an inner layer of thin polymeric material, an outer layer of polymeric material, and a central region comprising a coil, braid, stent, plurality of hoops, or other reinforcement. It is beneficial to create a bond between the outer and inner layers at a plurality of points, most preferably at the interstices or perforations in the reinforcement structure, which is generally fenestrated. Such bonding between the inner and outer layers causes a braided structure to lock in place. In another embodiment, the inner and outer layers are not fused or bonded together in at least some, or all, places. When similar materials are used for the inner and outer layers, the sheath structure can advantageously be fabricated by fusing of the inner and outer layer to create a uniform, non-layered structure surrounding the reinforcement. The polymeric materials used for the outer wall of the jacket are preferably elastomeric to maximize flexibility of the catheter. The polymeric materials used in the composite catheter inner wall may be the same materials as those used for the outer wall, or they may be different. In another embodiment, a composite tubular structure can be co-extruded by extruding a polymeric compound with a stent, braid, or coil structure embedded therein. The reinforcing structure is preferably fabricated from annealed metals, such as fully annealed stainless steel, titanium, or the like. In this embodiment, once expanded, the folds or crimps can be held open by the reinforcement structure embedded within the sheath, wherein the reinforcement structure is malleable but retains sufficient force to overcome any forces imparted by the sheath tubing.

In another embodiment of the invention, it is advantageous that the sheath comprise a radiopaque marker or markers. The radiopaque markers may be affixed to the non-expandable portion or they may be affixed to the expandable portion. Markers affixed to the radially expandable portion preferably do not restrain the sheath or catheter from radial expansion or collapse. Markers affixed to the non-expandable portion, such as the catheter shaft of a balloon dilator may be simple rings that are not radially expandable. Radiopaque markers include shapes fabricated from malleable material such as gold, platinum, tantalum, platinum iridium, and the like. Radiopacity can also be increased by vapor deposition coating or plating metal parts of the catheter with metals or alloys of gold, platinum, tantalum, platinum-iridium, and the like. Expandable markers may be fabricated as undulated or wavy rings, bendable wire wound circumferentially around the sheath, or other structures such as are found commonly on stents, grafts or catheters used for endovascular access in the body. Expandable radiopaque structures may also include disconnected or incomplete surround shapes affixed to the surface of a sleeve or other expandable shape. Non-expandable structures include circular rings or other structures that completely surround the catheter circumferentially and are strong enough to resist expansion. In another embodiment, the polymeric materials of the catheter or sheath may be loaded with radiopaque filler materials such as, but not limited to, bismuth salts, or barium salts, or the like, at percentages ranging from 1% to 50% by weight in order to increase radiopacity. The radiopaque markers allow the sheath to be guided and monitored using fluoroscopy.

In another embodiment of the invention, in order to enable radial or circumferential expansive translation of the reinforcement, it may be beneficial not to completely bond the inner and outer layers together, thus allowing for some motion of the reinforcement in translation as well as the normal circumferential expansion. Regions of non-bonding may be created by selective bonding between the two layers or by creating non-bonding regions using a slip layer fabricated from polymers, ceramics or metals. Radial expansion capabilities are important because the proximal end needs to transition to the distal expansive end and, to minimize manufacturing costs, the same catheter may be employed at both the proximal and distal end, with the expansive distal end undergoing secondary operations to permit radial or diametric expansion.

In another embodiment, the distal end of a catheter is fabricated using an inner tubular layer, which is thin and lubricious. This inner layer is fabricated from materials such as, but not limited to, FEP, PTFE, polyamide, polyethylene, polypropylene, Pebax, Hytrel, and the like. The reinforcement layer comprises a coil, braid, stent, or plurality of expandable, foldable, or collapsible rings, which are generally malleable and maintain their shape once deformed. Preferred materials for fabricating the reinforcement layer include but are not limited to, stainless steel, tantalum, gold, platinum, platinum-iridium, titanium, nitinol, and the like. The materials are preferably fully annealed or, in the case of nitinol, fully martensitic. The outer layer is fabricated from materials such as, but not limited to, FEP, PTFE, polyamide, polyethylene, polypropylene, polyurethane, Pebax, Hytrel, and the like. The inner layer is fused or bonded to the outer layer through holes in the reinforcement layer to create a composite unitary structure. The structure is crimped radially inward to a reduced cross-sectional area. A balloon dilator is inserted into the structure before crimping or after an initial crimping and before a final sheath crimping. The balloon dilator is capable of forced expansion of the reinforcement layer, which provides sufficient strength necessary to overcome any forces imparted by the polymeric tubing.

Another embodiment of the invention comprises a method of providing transluminal access. The method comprises inserting an endoscope into a patient, trans-esophageally, into the duodenum. Under direct optical visualization, fluoroscopy, MRI, or the like, a guidewire is passed through the instrument channel of the endoscope through the papilla sphincter and into the common bile duct or pancreatic duct. The guidewire is manipulated, under the visual control described above, into the bile duct or pancreatic duct through its exit into the duodenum. The guidewire is next advanced to the appropriate location within the bile duct or pancreatic duct. The eondoscope is next removed, leaving the guidewire in place. The transluminal access sheath is next advanced over the guidewire trans-esophageally so that its distal tip is now resident in the common bile duct or the pancreatic duct. The position of the guidewire is maintained carefully so that it does not come out of the ducts and fall into the duodenum. The removable dilator, which is removably affixed integrally inside the transluminal access sheath, comprises the guidewire lumen, and is used to guide, and maintain, placement of the access sheath into the urinary lumens.

In another embodiment of the invention, the expandable access sheath is configured to bend, or flex, around sharp corners and be advanced into the bile duct or pancreatic duct. Provision can optionally be made to actively orient or steer the sheath through the appropriate angles. The expandable sheath also needs to be able to approach the duct from a variety of positions. Expansion of the distal end of the access sheath from a first smaller diameter cross-section to a second larger diameter cross-section is next performed, using the balloon dilator. The balloon dilator is subsequently removed from the sheath to permit passage of instruments that would not normally have been able to be inserted into the bile or pancreatic duct due to the presence of strictures, stones, or other stenoses of carcinogenic or benign origin. The method further optionally involves releasing the elongate tubular body from a constraining tubular jacket, removing the expandable member from the elongate tubular body; inserting appropriate instrumentation, and performing the therapeutic or diagnostic procedure. Once the sheath is in place, the guidewire may be removed or, preferably, it may be left in place. The sphincter of hepatopancreatic ampulla is gently dilated with radial force, preferably to a diameter of 10 mm or less, rather than being cut open by a sphincterotomy procedure or translationally dilated by a tapered dilator or obturator. In one embodiment, the use of the expandable GI sheath eliminates the need for a large diameter right-angle endoscope in the main gastrointestinal tract with resultant benefits in reduced patient discomfort.

In another embodiment of the invention, further endoscopy and stone extraction may be performed with a forward-looking endoscope placed through the working channel of the expanded transluminal sheath. Endoscopes used in this embodiment can be much smaller (1 to 4 mm diameter) than standard endoscopes (generally 5 mm diameter or larger) since they do not require a working channel as that is contained within the sheath. Removed calculi or stones are fully withdrawn through the conduit of the sheath by graspers, a basket, a suction device, or the like. The stones can first be broken into smaller pieces using lasers, acoustic energy, or the like so that the pieces can be withdrawn into the sheath. The graspers may comprise jaws, basket traps, or the like. The sheath may optionally comprise a window or port in the region outside the sphincter, so that calculi, fluid, bile, irrigant, and debris can be discarded into the small intestine without the need to fully withdraw the graspers, basket, or suction device all the way out the proximal end of the sheath. The window or port can also comprise a closure that can be selectively operated to seal off the port when not in use or open the port when it is needed. The port or window can advantageously be denoted or surrounded by a radiopaque structure or marker to facilitate fluoroscopic monitoring. An advantage of the sheath of this configuration is its ability to provide a path for fluid, bile debris, blood, or other materials to be evacuated from the body lumen being accessed, whereas current systems may not offer such drainage channels. The sheath, dilator, or both can comprise multiple channels or lumens for these purposes. The sheath, in this and other embodiments, can be configured to maximize softness and resilience, especially in the area that traverses the thoracic region, since a stiff, non-resilient device may impinge, or generate pressure, on thoracic structures causing cardiopulmonary complications in the patient. The soft, compliant, resilient sheath is configured to comprise elements that provide for column strength and torqueability. In yet another embodiment, an inflatable balloon can be used to assist with tamponade or to slow or stop blood loss following therapy while coagulation occurs. In this embodiment, the balloon is affixed to the exterior of the sheath. The balloon is selectively located along the outside of the length of the sheath and can be optimally inflated to provide stability during the procedure. The balloon can also be affixed to a separate catheter slidably inserted through the sheath. Balloon inflation lumens are provided either in the catheter or as an annulus or lumen in the sheath. In another embodiment, the method comprises removal of the; sheath from the common bile duct or pancreatic duct at the end of the procedure. Finally, the procedure involves removing the elongate tubular body from the patient.

In another embodiment, the side-looking endoscope is advanced to the duodenum. The expandable transluminal access sheath is advanced through the working channel of the endoscope with its dilator in place. A guidewire, preferably an atraumatic guidewire, is advanced through the working channel of the endoscope into the common bile duct. The sheath is advanced into the common bile duct or pancreatic duct, over a guidewire, while the endoscope remains in the duodenum. The sheath is next expanded by action of the dilator. The expanded region of the sheath may now be larger than that part that is resident within the working channel of the endoscope and in the embodiment where expansion is not reversible, the expanded region of the sheath cannot be retracted within the working channel. The Sphincter of hepatopancreatic ampulla is dilated, preferably in a gentle fashion and over a period of time, with or without the need for a sphincterotomy. The guidewire may or may not be removed from the sheath and instrumentation inserted therethrough to a target site. Rapid exchange guidewire apparatus, and methodology to use the apparatus, are beneficially provided in conjunction with the sheath, its dilator, or both for this and all other embodiments. The rapid exchange guidewire exchange apparatus, including guidewire access ports within 12 inches of the distal or proximal end of the sheath, are capable of handling multiple guidewires and multiple catheters being placed over said guidewires. Manipulation of each of the guidewires separately is preferably permitted by the sheath configuration. Following any therapeutic or diagnostic procedures, the sheath and side-viewing endoscope are removed from the patient, separately, or as a unit.

In another embodiment, the expandable transluminal access sheath is inserted through a side-looking endoscope and advanced over a guidewire into the common bile duct or the pancreatic duct. The sheath is next dilated radially by means of an internal dilator, preferably a balloon dilator. A portion of the distal section of the sheath is then detached from its more proximal region. The balloon dilator is removed from the sheath by withdrawing proximally. In one embodiment, expansion of the dilator can be used as the mechanism to generate the detachment force on the distal end of the sheath. The endoscope, proximal sheath section, and guidewire are removed from the patient leaving the expanded sheath within the bile duct or pancreatic duct to serve as a stent. The portion of the sheath remaining within the patient following separation may project through the sphincter of hepatopancreatic ampulla or it may reside inside thus retaining sphincteric function, depending on the pathology (or lack of pathology).

In one embodiment, where the transluminal access sheath is used to provide access to the biliary or pancreatic ducts, the access sheath may be used to provide access by tools adapted to perform biopsy, stone extraction, stent placement, or resection of transitional cell carcinoma and other diagnostic or therapeutic procedures. Other applications of the transluminal access sheath include a variety of diagnostic or therapeutic clinical situations, which require access to the inside of the body, through either an artificially created, percutaneous access, or through another natural body lumen.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a front view schematic representation of the human digestive tract including the esophagus, the stomach, the duodenum, the liver, and the pancreas;

FIG. 2 is a schematic cross-sectional representation of the duodenum, the common bile duct and the pancreatic duct;

FIG. 3 is a schematic cross-sectional representation of the duodenum, the pancreatic duct, and the common bile duct shown with a cutaway of the wall, further with stones in the common bile duct;

FIG. 4 is a cross-sectional illustration of the duodenum, the common bile duct, and the pancreatic duct with stones in the common bile duct with a side-viewing endoscope placed within the duodenum and a guidewire advanced into the common bile duct, according to an embodiment of the invention;

FIG. 5 illustrates a side view of a gastric, radially expandable, collapsed, transluminal sheath, inserted into the common bile duct over the guidewire following removal of the endoscope, according to an embodiment of the invention;

FIG. 6 illustrates a side view of the gastric, radially expandable transluminal sheath following expansion of its distal portion by an internal dilator, according to an embodiment of the invention;

FIG. 7 is an illustration of the gastric, radially expandable transluminal sheath with the dilator having been removed, according to an embodiment of the invention;

FIG. 8 illustrates a side view of the gastric, radially expandable sheath wherein and endoscope with graspers is advanced through the sheath and is removing a stone, following fragmentation, according to an embodiment of the invention;

FIG. 9 illustrates a side view of a collapsed, radially expandable sheath having been inserted through the working channel of an endoscope into the common bile duct, according to an embodiment of the invention;

FIG. 10 illustrates a side view of a radially expandable sheath having been inserted through the working channel of an endoscope into the common bile duct and expanded with graspers extended therethrough, according to an embodiment of the invention;

FIG. 11 illustrates a side view of a radially expandable sheath having been inserted through the working channel of an endoscope into the common bile duct with the entire assembly being withdrawn into the descending duodenum to remove a stone, according to an embodiment of the invention;

FIG. 12 illustrates a side view of a collapsed, radially expandable, detachable sheath having been inserted through the working channel of an endoscope into the common bile duct, according to an embodiment of the invention;

FIG. 13 illustrates a side view of a radially expandable, detachable sheath having been inserted through the working channel of an endoscope into the common bile duct and then expanded by its internal dilator, according to an embodiment of the invention;

FIG. 14 illustrates a side view of an expanded radially expandable, detachable sheath following removal of the deflated balloon dilator and detachment from the proximal portion of the sheath, according to an embodiment of the invention;

FIG. 15 illustrates a side view of an expanded radially expandable, detachable sheath having been inserted into the common bile duct, and detached from its proximal portion, which has been removed from the patient, leaving the stent fully within the common bile duct and not projecting through the sphincter, according to an embodiment of the invention;

FIG. 16 illustrates a radially expandable sheath having been inserted into the common bile duct, said sheath further comprising a window or port for disposal of debris, according to an embodiment of the invention;

FIG. 17 illustrates a radially expandable sheath, wherein the sheath has an opening on one side to accommodate flow from the pancreatic duct, according to an embodiment of the invention;

FIG. 18A illustrates a side view of a collapsed, non-expanded sheath, according to an embodiment of the invention;

FIG. 18B illustrates a side view of an expanded sheath, according to an embodiment of the invention, and

FIG. 18C illustrates a side view of an expanded sheath with the dilator removed, according to an embodiment of the invention.

### DETAILED DESCRIPTION OF THE PREFERRED EMBODIMENTS

The invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

The disclosed embodiments, which are generally termed a catheter or a sheath, can be described as being an axially elongate hollow tubular structure having a proximal end and a distal end. Such tubular structures are generally shown as having a round or circular cross-section . However, it should be appreciated that the cross-section can have other shapes. The axially elongate structure further has a longitudinal axis and has an internal through lumen that preferably extends from the proximal end to the distal end for the passage of instruments, fluids, tissue, or other materials. The axially elongate hollow tubular structure is generally flexible and capable of bending, to a greater or lesser degree, through one or more arcs in one or more directions perpendicular to the main longitudinal axis. As is commonly used in the art of medical devices, the proximal end of the device is that end that is closest to the user, typically a gastroenterologist, surgeon, or interventionalist. The distal end of the device is that end closest to the patient or that is first inserted into the patient. A direction being described as being proximal to a certain landmark will be closer to the surgeon, along the longitudinal axis, and further from the patient than the specified landmark. The diameter of a catheter is often measured in “French Size” which can be defined as 3 times the diameter in millimeters (mm). For example, a 15 French catheter is 5 mm in diameter. The French size is designed to approximate the circumference of the catheter in mm and is often useful for catheters that have non-circular cross-sectional configurations. While the original measurement of “French” used pi (3.1415 . . . ) as the conversion factor between diameters in mm and French, the system has degraded today to where the conversion factor is exactly 3.0.

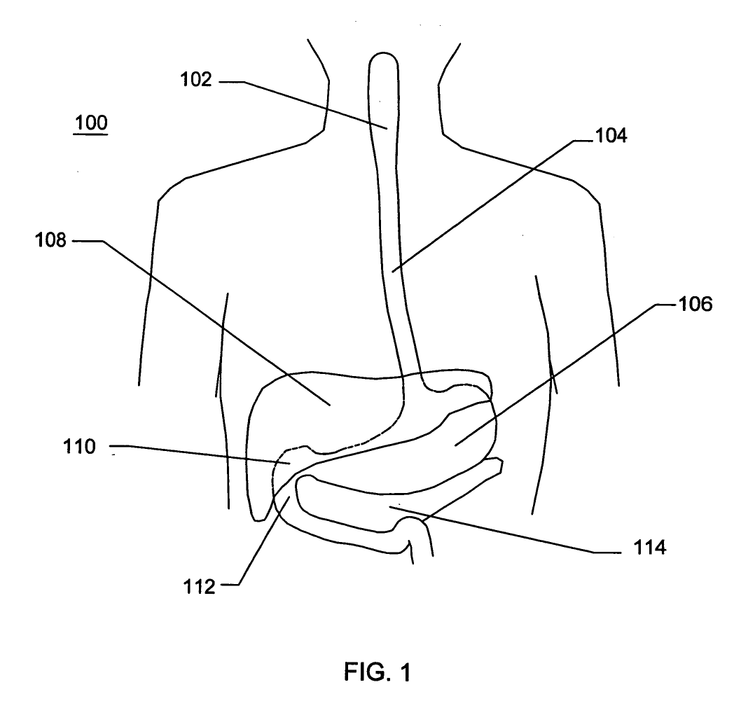

FIG. 1 is a schematic frontal illustration (anterior view) of a human patient 100 comprising a pharynx 102, a esophagus 104, a stomach 106, a liver 108, a superior duodenum 110, a descending duodenum 112, and a pancreas 114. In this illustration, the left anatomical side of the body of the patient 100 is toward the right of the illustration.

Referring to FIG. 1, the pharynx 102 is a chamber in the throat of the patient 100 that is operably connected to the mouth (not shown) and nose (not shown) with further access to the trachea (not shown) and the esophagus 104. Generally, the internal surfaces of the esophagus 104, the stomach 106, and the duodenum 110 and 112 comprise smooth muscle that exhibits a peristaltic motion to move food through the system.

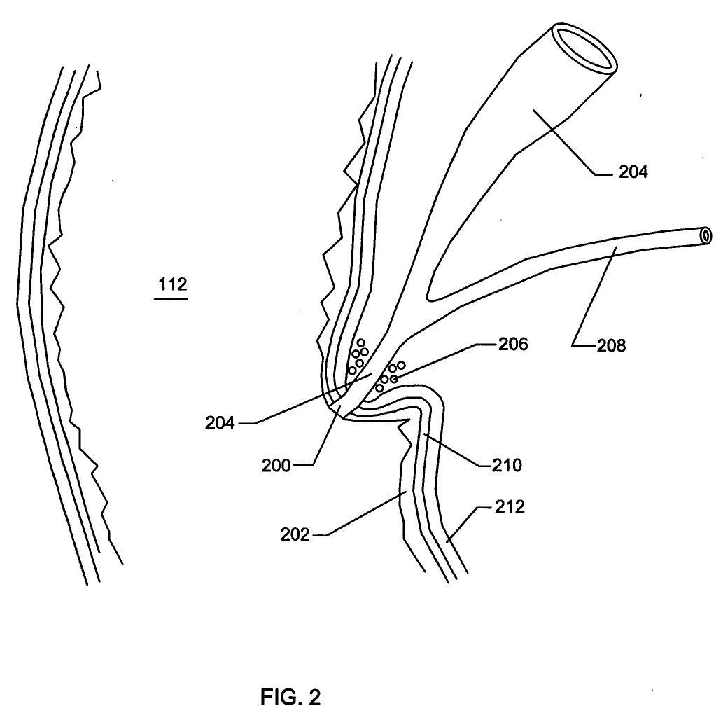

FIG. 2 is a schematic frontal illustration, looking posteriorly from the anterior side, of the descending duodenum 112. The walls of the duodenum 112 comprise an outer longitudinal layer and an inner circular layer of smooth muscle 210 and are internally lined with submucosa 202 further comprising duodenal, or Brunner's, glands. Branching from the descending duodenum 112, at the major duodenal papilla, also known as the ampulla of Vater, 200, is the common bile duct 204, and the side-branching pancreatic duct 208. The muscular valving structure surrounding the major duodenal papilla 200 is the sphincter of hepatopancreatic ampulla, also known as the sphincter of Oddi, 206. In this illustration, the left anatomical side of the body is toward the right of the illustration.

Referring to FIG. 2, the sphincter of hepatopancreatic ampulla 206 permits material to exit the common bile duct 204 into the lumen of the descending duodenum 112 when digesting food is present, but prevents migration of fecal or gastric material retrograde into the common bile duct 204 or the pancreatic duct 208. Referring to FIGS. 1 and 2, the common bile duct 204 serves as the main drainage channel for the gall bladder (not shown), the liver 108, and the pancreas 114. Any damage to the sphincter of hepatopancreatic ampulla 206 could result in infection of the aforementioned drainage source organs. The angle of the common bile duct 204 relative to the lumen of the descending duodenum 112 is shown as being approximately 120 degrees but the angle could vary between approximately 90 to 180 degrees. Furthermore, anatomical variants on the structure include circumstances where the pancreatic duct 208 and the common bile duct 204 enter the descending duodenum 112 through separate orifices in the major duodenal papilla 200. Other configurations include those where they come together or branch just at the entrance to the major duodenal papilla 200 or where they branch a measurable distance upstream of the papilla 200, the lafter anatomy being the one illustrated in FIG. 2.

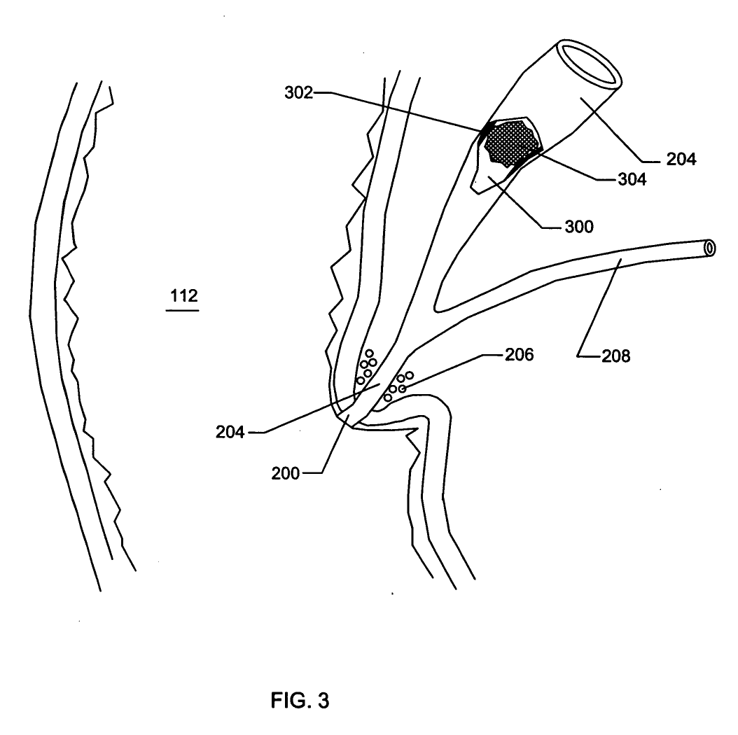

FIG. 3 is a frontal illustration, looking posteriorly from the anterior side, of the descending duodenum 112. Branching from the descending duodenum 112, at the major duodenal papilla 200, is the common bile duct 204, and the side-branching pancreatic duct 208. The sphincter of hepatopancreatic ampulla 206 is also shown. Further illustrated is a cutaway view of the common bile duct 204 showing the internal lumen 300, the wall 302, and a stone 304 lodged therein.

Referring to FIG. 3, the stone 304 is generally composed of cholesterol, calcium salts, or similar materials. The stone 304 forms in the common bile duct 204, the pancreatic duct 208 or one of the other branch ducts of the common bile duct 204. The stone 304 can migrate or lodge in the duct causing blockage, pain, infection, and the like. Such stones 304 may range in size up to 10-cm or larger and removal is often necessary. Removal of large stones through the common bile duct 204 may require dilation of the duct, dilation or surgical incision of the sphincter of hepatopancreatic ampulla 206, or both. Removal of large stones may also entail breaking up such stones 304 using methods such as, but not limited to, high-frequency focused ultrasound, acoustic waves, radio-frequency energy, mechanical energy, light energy such as that derived from lasers, and the like.

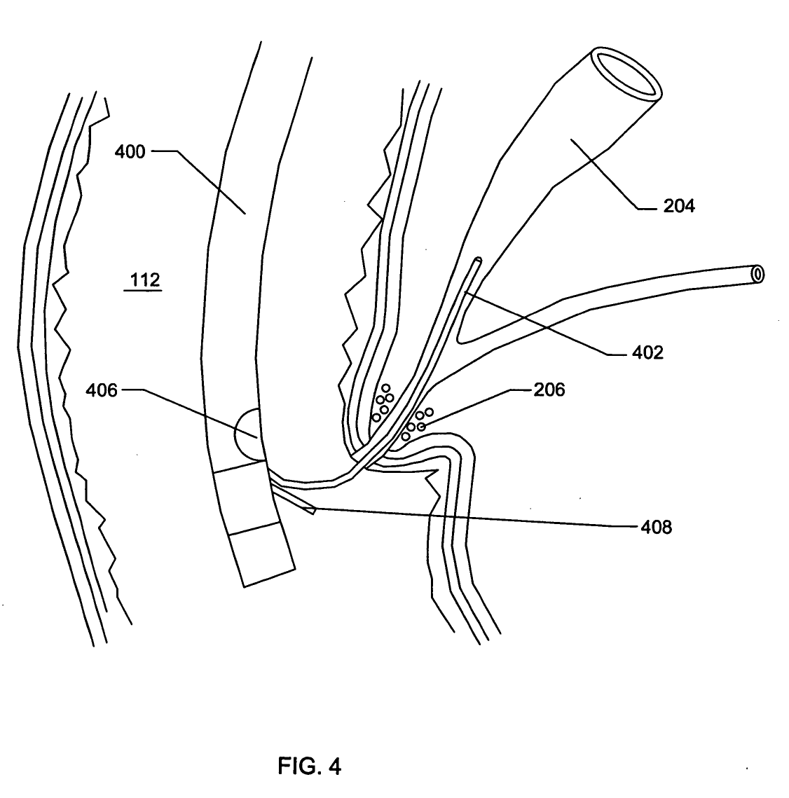

FIG. 4 is a cross-sectional illustration of the descending duodenum 112, the sphincter of hepatopancreatic ampulla 206, and the common bile duct 204. A side-viewing endoscope 400 is placed within the duodenum 112 and a guidewire 402 advanced into the common bile duct 204 through the sphincter 206. The endoscope 400 further comprises a side viewing lens 406 and a tool deflecting mechanism 408. The endoscope 400 may further comprise internal scope deflection mechanisms to facilitate navigation of tortuous anatomy.

Referring to FIG. 4, the endoscope 400 has on outside diameter of approximately 15 French of 5 millimeters. The endoscope 400 may further comprise a working channel (not shown), an optical telescope element (not shown), a light source channel (not shown), and an internal optional deflection mechanism (not shown). The side-viewing lens 406 is located at the distal end of the optical telescope element and may also comprise the distal end of the light source channel. The deflecting mechanism 408 may be stationary, such as an angled or curved surface, or it may be actuable from the proximal end of the end oscope 400 by way of a control rod or wire and a lever, the latter being affixed at or near the proximal end of the endoscope 400. The deflecting mechanism 408 is located at the distal end of the working channel, which currently holds the guidewire 402 and which may ultimately also carry a catheter for therapy or diagnosis. The guidewire 402 has been advanced and turned sidewise by the deflecting mechanism 408. Referring to FIGS. 2 and 4, the guidewire 402 has been inserted through the papilla 200 and into the common bile duct 204.

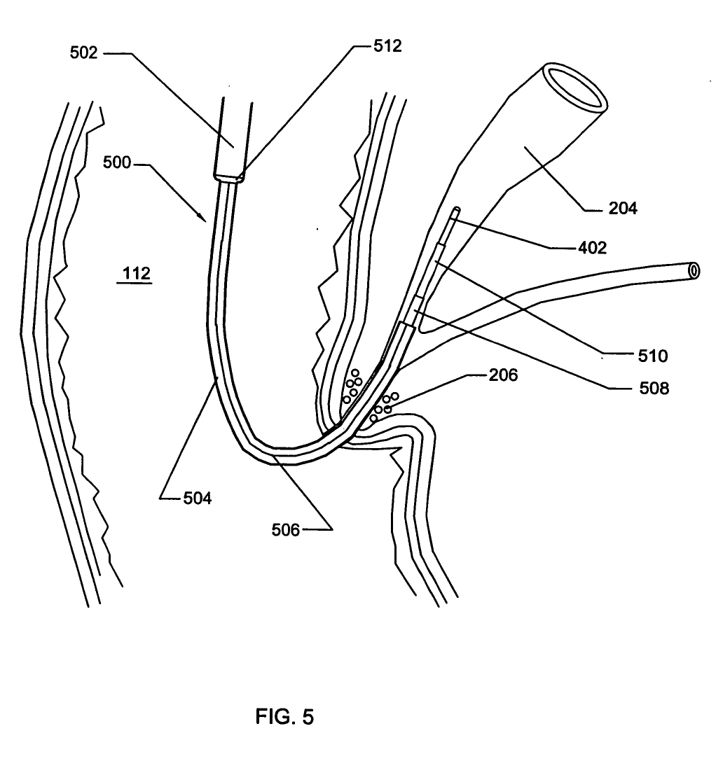

FIG. 5 is a cross-sectional illustration of the descending duodenum 112, the sphincter of hepatopancreatic ampulla 206, and the common bile duct 204. An expandable access sheath 500 is placed over the guidewire 402, following removal of the endoscope 400 (refer to FIG. 4) and advanced into the common bile duct 204 through the sphincter of hepatopancreatic ampulla 206. The sheath 500 further comprises a proximal non-expandable region 502, a transition zone 512, a distal expandable region 504, an expansion fold 506, a dilatation balloon 508, a dilator shaft 510, a sheath hub (not shown) and a dilator hub (not shown).

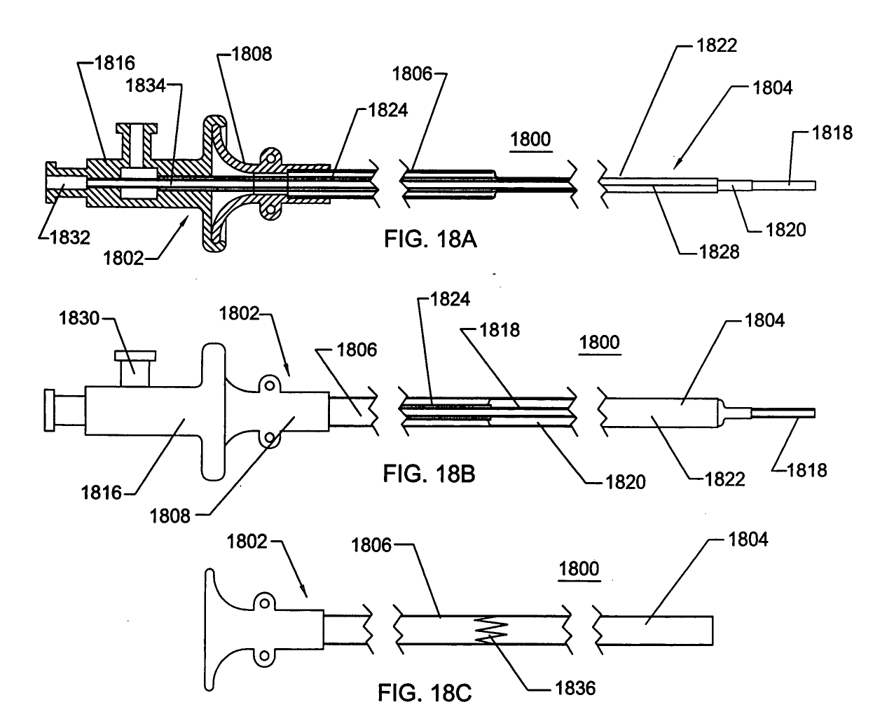

Referring to FIG. 5, an expandable access sheath 500 having certain features and advantages is shown is pre-assembled with its internal dilator. An embodiment of the sheath will be described in more detail with reference to FIGS. 18A-C The internal dilator comprises the dilatation balloon 508, the dilator shaft 510, and the dilator hub. The internal dilator uses multi-lumen tubing, coaxial multiple tubes, or the like to allow for guidewire 402 passage through a guidewire lumen (not shown) and for the inflation and deflation of the balloon 508, which is located near the distal end of the dilator. The balloon 508 inflation is accomplished through a port in the dilator hub (not shown) located at the proximal end of the dilator. An inflation device such as those commercially available in the medical device business and comprising a syringe, a mechanical advantage driver, and an optional pressure gauge, is affixed to the dilator hub by way of a pressure line with a luer, or other, fitting. The deflated balloon 508 is folded to form wings and inserted inside the distal sheath expandable region 504. The deflated balloon 508 traverses the longitudinal extents of the expandable region 504 and its position is determined by the relationship, preferably locking, between the dilator hub and the sheath hub. The expandable region 504 is folded down over the deflated balloon 508 in such a way that one or more longitudinally oriented folds 506 are created on the expandable region 504. The expandable region 504 is now diametrically compressed. and is substantially smaller than the proximal non-expandable region 502 of the sheath 500. The expandable region 504, mounted over the dilator, which is slidably disposed over the guidewire 402, can be advanced through small orifices such as the sphincter of hepatopancreatic ampulla 206. This sheath-dilator structure 500 is very flexible and can turn sharp corners. The expandable region 504 is constructed as a composite structure with a malleable reinforcement embedded within a flexible, thin-wall polymer tube. The thin-wall polymer tube exerts insubstantial force relative to the malleable reinforcement so the configuration of the malleable reinforcement controls the configuration of the surrounding polymer. Thus, in this embodiment, the folded expandable region 504 stays folded, without the need for an outer compression jacket, until such time as the structure is expanded.

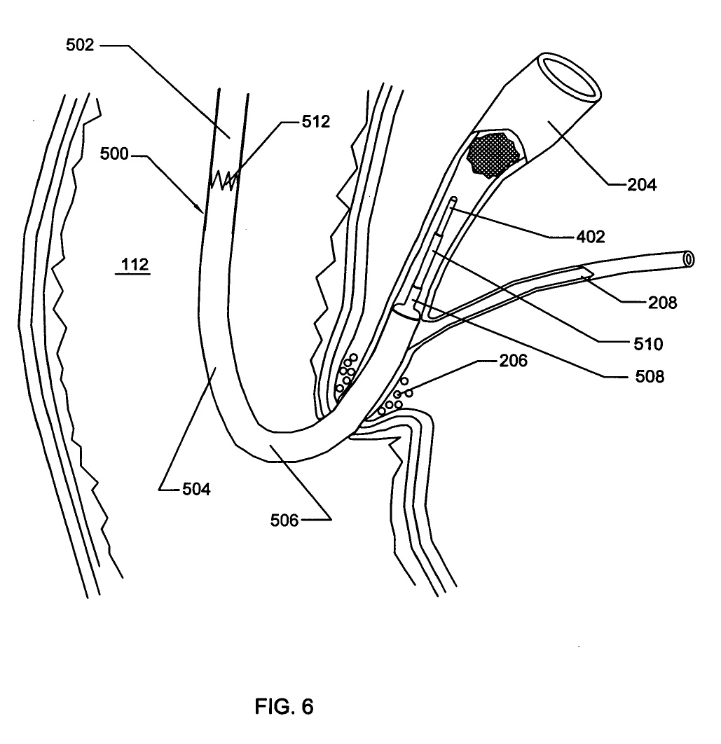

FIG. 6 illustrates a side view of the radially expandable transluminal sheath 500 following expansion of its distal portion 504 by its internal dilator. The non-expandable region 502 and the transition region 512 the sheath 500 are both resident in the descending duodenum 112. The expandable region 506 has turned through an angle and the distal end of the sheath 500 with its internal dilator are both resident in the common bile duct 204. The balloon 508 has been expanded under pressure from a liquid-filled external inflation device (not shown) operably connected to the inflation port (not shown) on the dilator hub (not shown) at the proximal end of the dilator tubing 510. The expandable region 504 has expanded diametrically and has dilated the sphincter of hepatopancreatic ampulla 206. The sphincter 206 is sealed by the sheath 500 so that no intestinal material can flow retrograde back into the common bile duct 204. The longitudinal fold 506, shown in FIG. 5, is no longer visible since the fold 506 has been dilated. The distal end of the expandable region 504 is resident in the common bile duct 204 just upstream of the bifurcation where the pancreatic duct 208 joins the common bile duct 204.

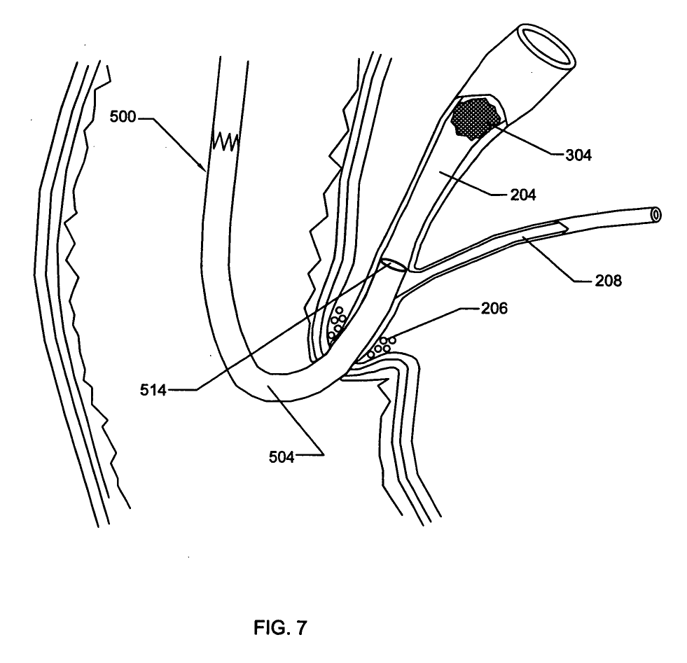

FIG. 7 is an illustration of the gastric, radially expandable transluminal sheath 500 with the guidewire 402, and the dilator, further comprising the balloon 508 and the dilator tubing 510, all shown on FIG. 6, having been removed leaving only the expandable region 504 in the common bile duct 204. The expandable region 504 continues to seal the sphincter 206. The stone 304 is now approachable from instrumentation inserted through the central lumen 514 of the sheath 500. In another embodiment, the sheath 500 can comprise, on its outer surface, devices for the performance of a sphincterotomy of the sphincter of Oddi 206. The sphincterotomy devices can include electrocautery instruments, sharp blades, wires, or the like. The blades can be actuated from the proximal end of the sheath 500 and made to open up to cut radially outward into the sphincter 206. The blades can further be sheathed or covered, the sheathing selectively withdrawn to expose the blades to the tissue so that the sphincterotomy can be performed. The wires or electrocautery elements can be electrically charged by a power supply at the proximal end of the sheath 500. By performing a sphincterotomy prior to, during, or just after the dilation, caused by sheath expansion, the maintenance of post-procedural sphincter function and the minimization of pancreatitis can be achieved. Dilatation of the sphincter of Oddi 206 with large diameter balloons has been suggested as the cause of increased risk of post-ERCP pancreatitis. The sheath 500 is configured so that it does not dilate the sphincter of Oddi 206 to a diameter greater than 10 mm and, preferably, not greater than 6 to 8 mm diameter. By minimizing the diameter of the dilation, the muscles actuating the sphincter of Oddi are preserved, the sphincter function is preserved following the ERCP, and reflux of contaminants into the pancreatic duct and ensuing pancreatitis are minimized. In order to remove large stones, which can be as large as 20 to 40 mm in their largest dimension, it is preferable to break these stones into smaller fragments through previously described lithotripsy methodology.

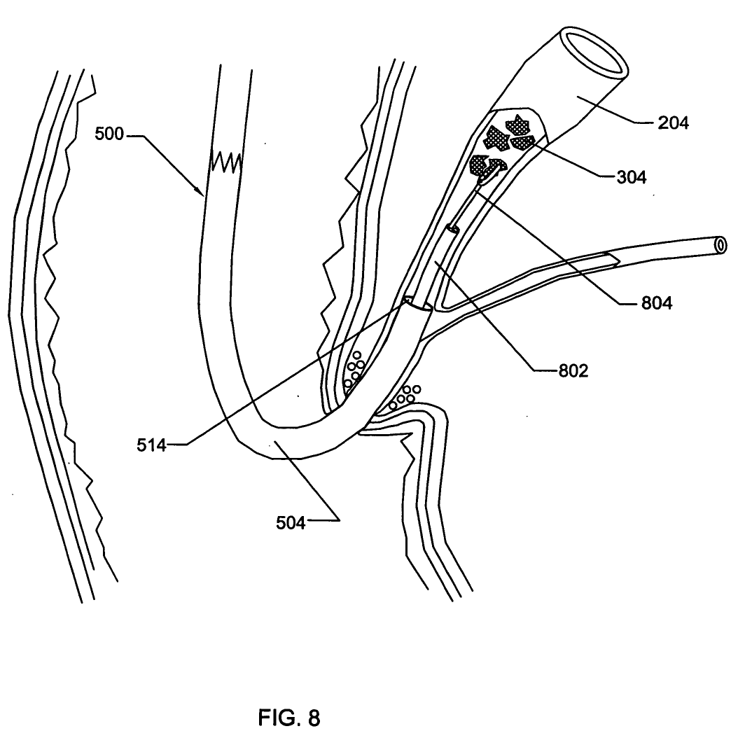

FIG. 8 illustrates a side view of the gastric, radially expandable sheath 500 wherein an endoscope 802, with graspers 804 inserted through the central lumen 514, is advanced through the sheath and is removing a stone 304, following fragmentation of the stone 304 into smaller pieces. The pieces of stone 304 reside in the common bile duct 204, as does the distal expandable end 504 of the sheath 500. Note that the graspers 804 can be larger than the endoscope 802 and its instrumentation channel because the entire assembly is now passed through and protected from damaging tissue by the sheath 500. Such a configuration, which is advantageous in removing large stones 304, cannot be used without sheath 500. The endoscope 802 is preferably a forward viewing endoscope with associated fiber optic bundles and light channels for illumination of the field. The endoscope can further comprise an irrigation channel or it can irrigate through the instrumentation channel.

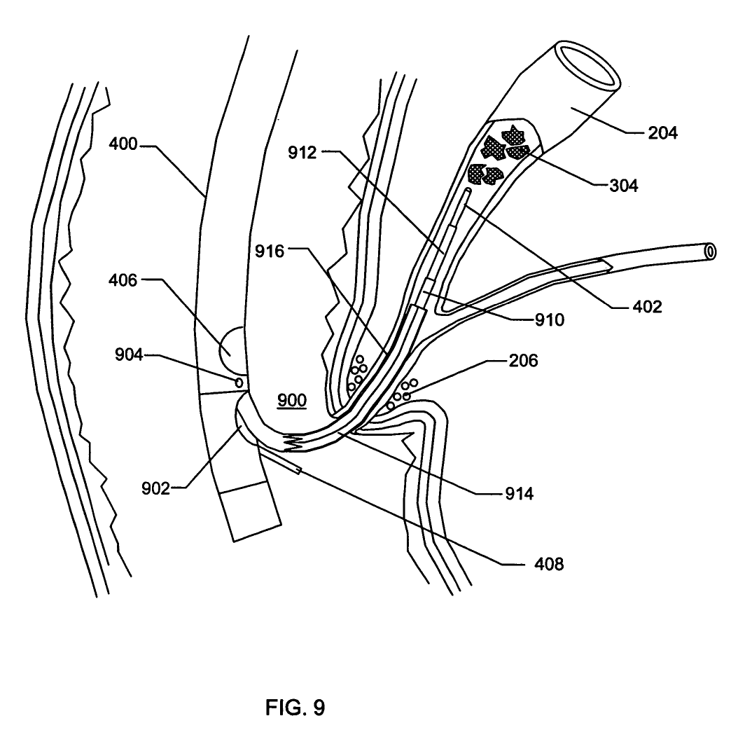

FIG. 9 illustrates a side view of a collapsed, radially expandable sheath 900 having been inserted through the working channel 902 of an endoscope 400 into the common bile duct 204. The endoscope 400 further comprises an instrument deflector 408 and a viewing lens 406, along with a light channel 904. The sheath 900 further comprises a distal region 916 comprising longitudinal folds or creases 904, a dilator balloon 910, and a dilator shaft 912. The sheath 900 is routed into the common bile duct 204 over the guidewire 402. The sheath 900 further projects through the sphincter of Oddi 206, at the entrance to the common bile duct 204. The sphincter 206 is only slightly dilated by the sheath 900, at this point, since the sheath is still in its compressed configuration. A plurality of calculi 304 can reside within the common bile duct 204 and impede drainage therefrom.

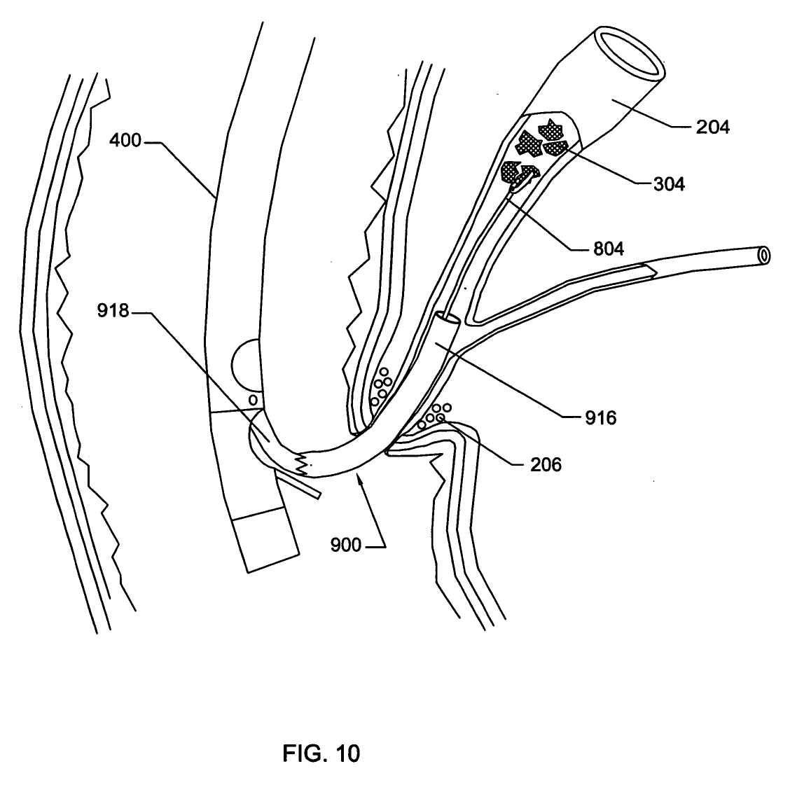

FIG. 10 illustrates a side view of the radially expandable sheath 900 having been inserted through the working channel of the endoscope 400 into the common bile duct 206 and the distal sheath region 916 has been diametrically expanded. Referring to FIG. 9, the dilator balloon 910 and the dilator shaft 912 have been withdrawn from the sheath 900. The longitudinal fold 914 has been expanded and is no longer visible in FIG. 10. The proximal region 918 of the sheath 900 is non-expandable and continues to reside within the endoscope 400. A pair of graspers 804 extends beyond the distal region 916 of the sheath 900. At this point, the distal region 916 is too large in diameter to be withdrawn into the endoscope 400 but the graspers 804 can be fully withdrawn or inserted.

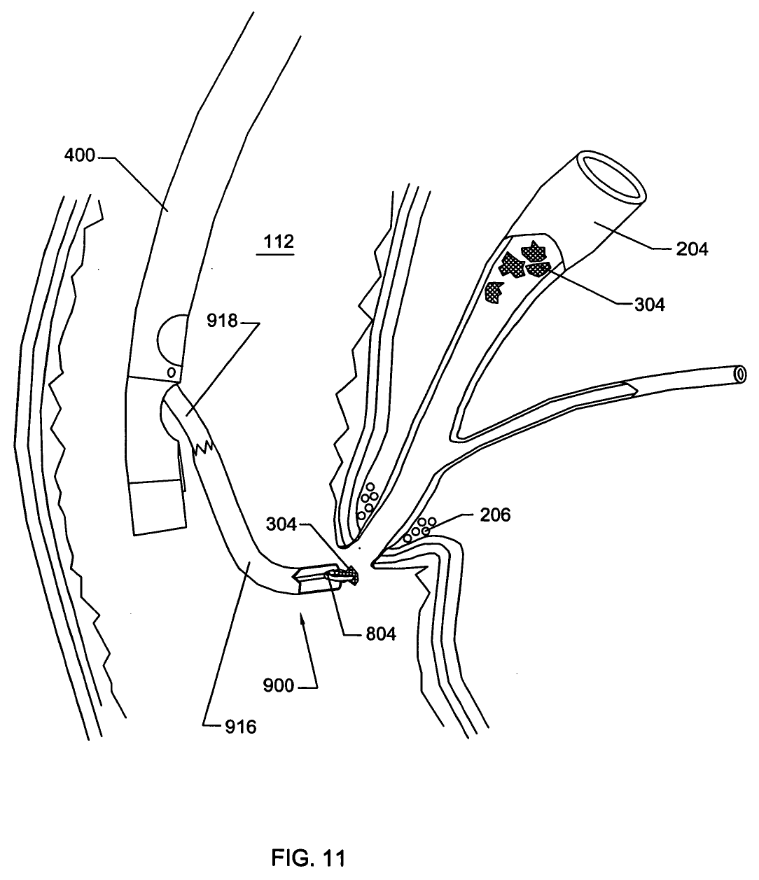

FIG. 11 illustrates a side view of the radially expandable sheath 900 having been inserted through the working channel of the endoscope 400 into the common bile duct 204 with the entire assembly being withdrawn into the descending duodenum 112 to remove a stone 304. The distal end of the expandable region 916 is shown in cutaway rendition revealing the graspers 804. The non-expandable proximal region 918 emerges from the endoscope 400. The entire endoscope 400 and sheath 900 is being withdrawn to remove the stone 304.

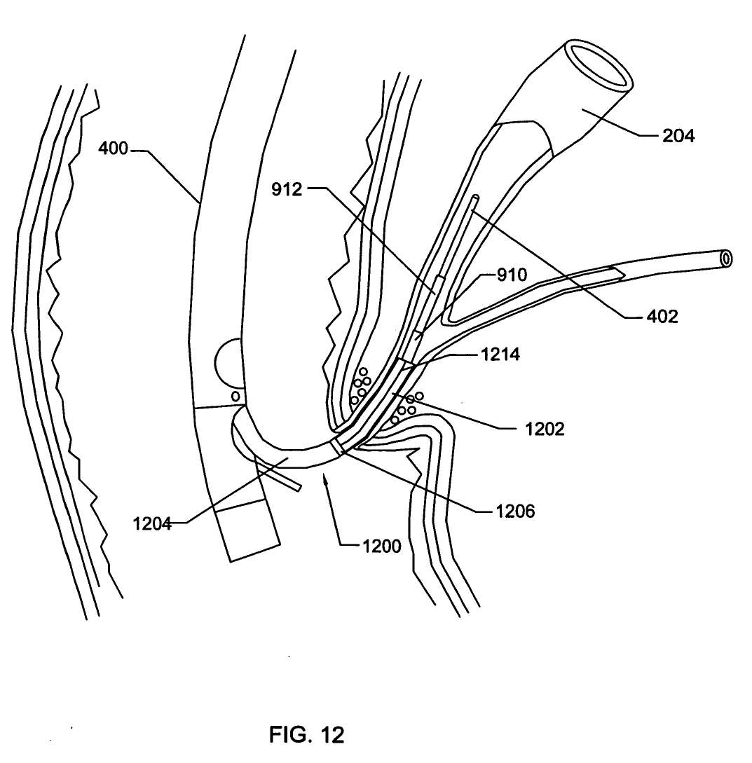

FIG. 12 illustrates a collapsed, radially expandable, detachable sheath 1200 having been inserted through the working channel of an endoscope 400 into the common bile duct 204. The proximal non-expandable region 1204 is releasably affixed to the distal expandable region 1202 by the releasable coupler 1206. The distal expandable region 1202 comprises one or more longitudinal folds or creases 1214. The sheath 1200 is coaxially, and slidably, connected to the dilator balloon 910, which is affixed and operably connected to the dilator shaft 912 so that the balloon 910 can be inflated through a lumen or annulus (not shown) extending from the proximal end of the dilator (not shown). The entire assembly is slidably engaged over, and tracks, the guidewire 402. The releasable coupler 1206 can be operably connected to an actuator (not shown) at the proximal end of the sheath 1200.

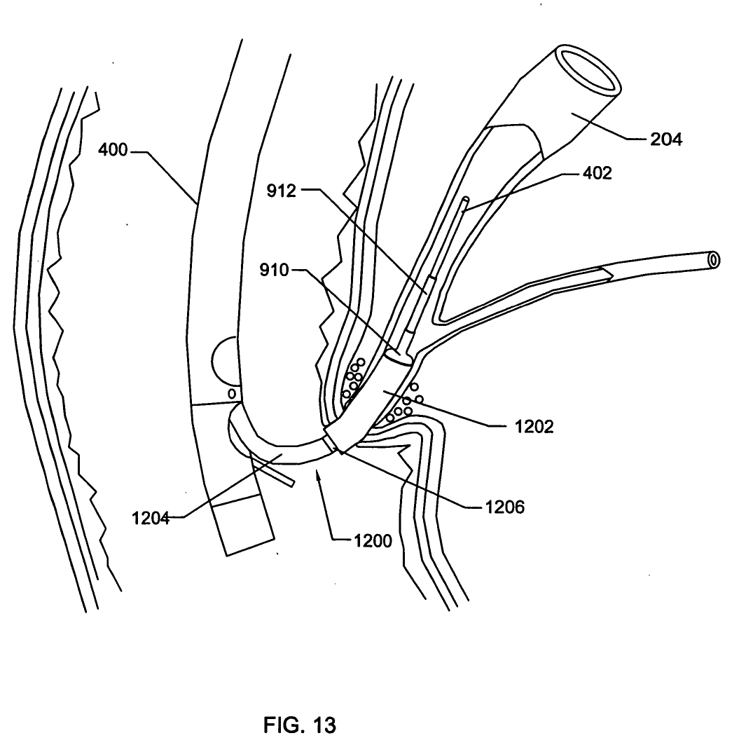

FIG. 13 illustrates the radially expandable, detachable sheath 1200 having been inserted into the common bile duct 204 and then expanded by its internal dilation balloon 910. The balloon 910 is affixed to the dilator shaft 912, which further comprises a central lumen for tracking over the guidewire 402. The sheath 1200 comprises the proximal non-expandable region 1204, the distal expandable region 1202, and the releasable coupler 1206. The proximal portion 1204 of the sheath 1200 extends through the working channel of the endoscope 400.

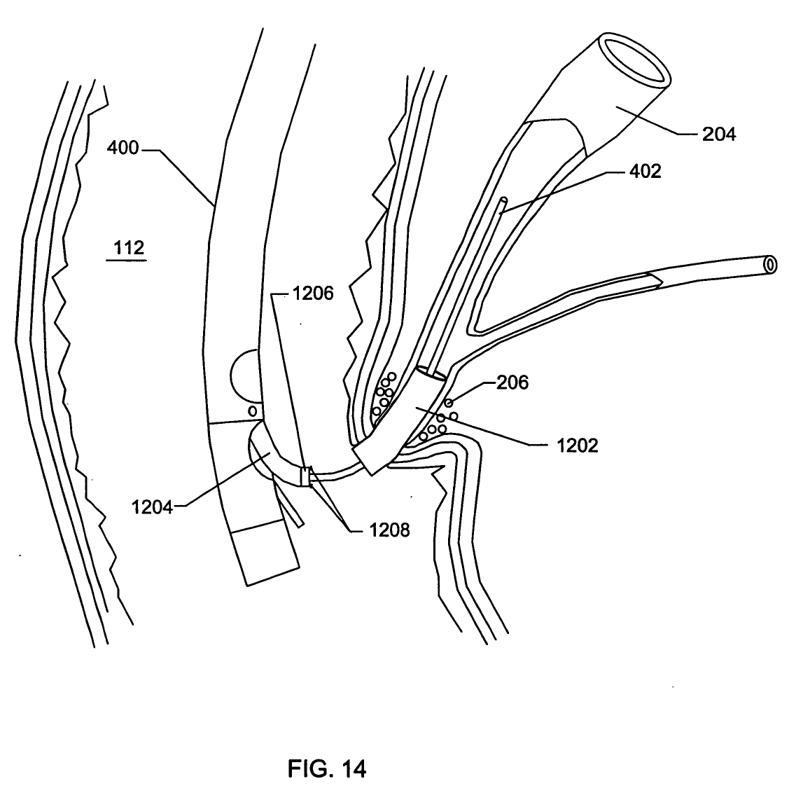

FIG. 14 illustrates the radially expandable, detachable sheath 1200 following removal of the deflated dilator balloon 910 and dilator shaft 912 and detachment of the expandable region 1202 from the proximal portion 1204 of the sheath 1200. The guidewire 402 remains in place in the common bile duct 204. Detachment of the expandable region 1202 from the proximal portion 1204 occurs at the coupler 1206. The coupler 1206 can be passive and release the expandable region 1202 when the balloon 910 is inflated, or in other embodiments, can be released by pull-wires, push-wires, or actuators powered by electrical, pneumatic, hydraulic, magnetic, light, heat, microwave, radio frequency, or other similar type of power. The coupler 1206 can use releasable latches 1208, zippers, clips, undercuts, or other mechanical interference to create the reversible coupling. In this illustration, the proximal portion 1204 is being withdrawn back into the descending duodenum 112. Once released from the proximal portion 1204, the expandable, releasable, distal region 1202 can reside within the anatomy and serve the function of a stent, a sphincter dilator, a sheath to facilitate further instrumentation, or a combination of the aforementioned. The guidewire 402, shown traversing the gap between the coupler 1206 and the expandable releasable distal region 1202 is removed at the appropriate time. Situations that may require such a device include those where a carcinoma has constricted the common bile duct or pancreatic duct and where long-term palliative relief of the obstruction is indicated without the trauma of a surgical intervention.

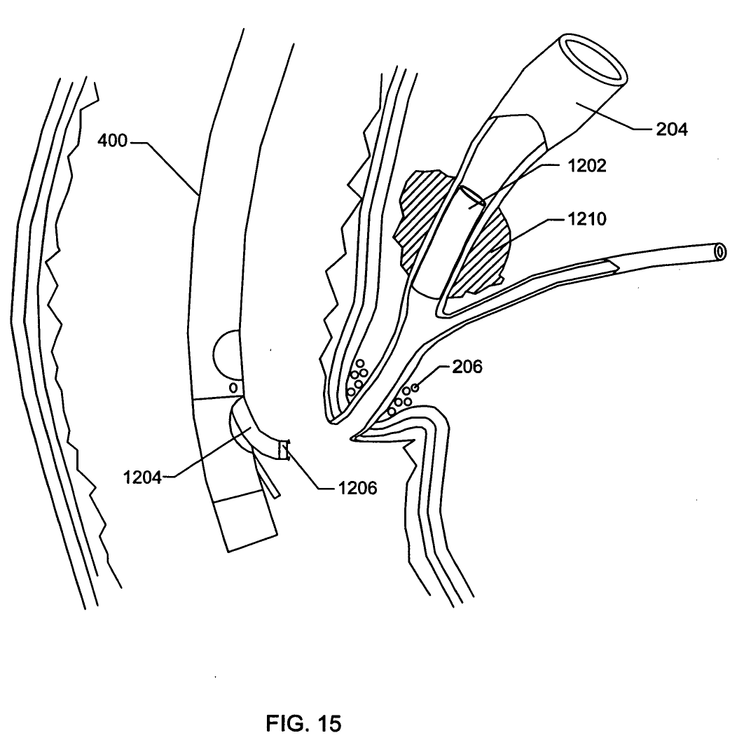

FIG. 15 illustrates a radially expandable, detachable sheath distal section 1202 having been inserted into the common bile duct 204, and detached from its proximal portion 1204 (Reference FIG. 14). The proximal portion 1204 has been removed from the patient, along with the coupler 1206, leaving the distal sheath section 1202 fully within the common bile duct 204 and not projecting through the sphincter 206. The distal sheath section 1202 serves the same function as a biliary stent and, in this case, is relieving a stenosis caused by a tumor 1210, which surrounds and constricts the common bile duct 204.

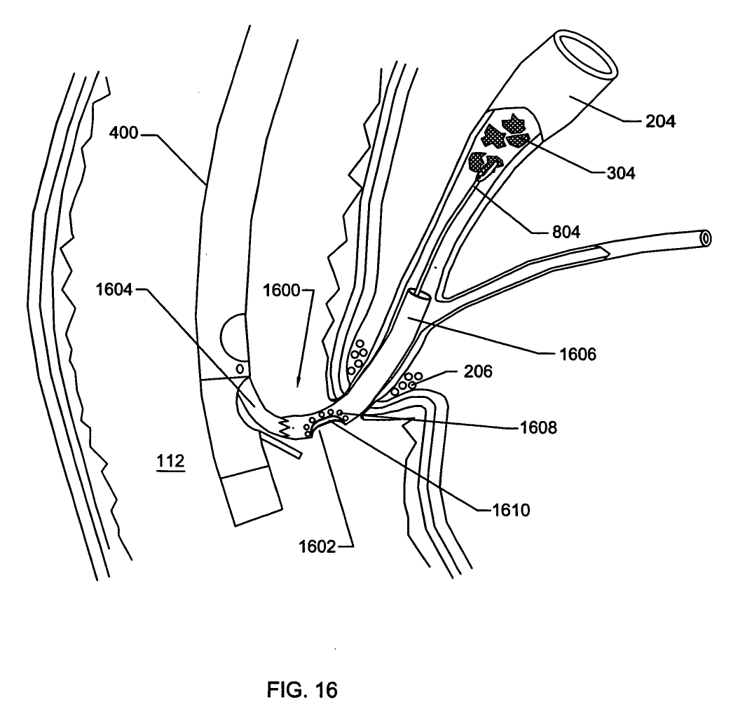

FIG. 16 illustrates a radially expandable sheath 1600 having been inserted into the common bile duct 204, said sheath 1600 further comprising a window or port 1602 for disposal of debris, such as calculi 304. The window is an opening that operably connects the inner lumen 1610 of the sheath 1600 to the environment outside the sheath 1600. In an embodiment, the window 1602 opens into the descending duodenum 112 through the wall of the distal expandable sheath region 1606. The proximal non-expandable region 1604 can be withdrawn into the endoscope 400 but the distal region 1606 is too large for such withdrawal. However, the lumen 1610 of the distal region 1606 is capable of holding the graspers 804 and calculi 304 of sufficiently small size. The window or opening 1602 preferably is as wide as the diameter of the sheath 1600 in the region where the window 1602 is placed. In this configuration, the sheath expandable region 1606 dilates and protects the sphincter 206 from damage and allows for instrument passage therethrough. The window or opening 1602 can comprise radiopaque markers 1608 to facilitate location and to denote the location and extents of the window 1602 during fluoroscopic monitoring.

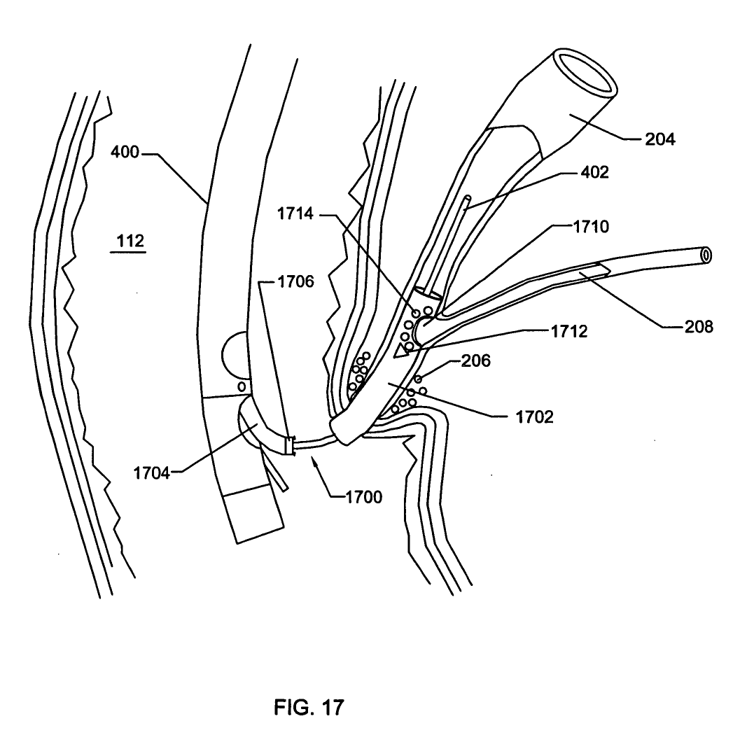

FIG. 17 illustrates a radially expandable sheath 1700, wherein the sheath 1700 comprises an expandable, releasable distal region 1702, a non-expandable proximal region 1704, a releasable coupler 1706, and a flow window 1710 to accommodate flow from the pancreatic duct 208. The distal region 1702 is shown expanded, and it resides in the common bile duct 204. The sheath 1700 is placed over a guidewire 402 and through the working channel of an endoscope 400, which is located in the descending duodenum 112. The sheath distal region 1702 extends through the sphincter of hepatopancreatic ampulla 206 and holds the sphincter open, in this embodiment. The distal region 1702 further comprises asymmetric radiopaque markers 1712 to provide rotational and longitudinal orientation information. The radiopaque markings 1712 are advantageously asymmetric and capable of providing rotational position information when their image or shadow is projected onto a two-dimensional plane. The radiopaque markings 1712 are fabricated from tantalum, platinum, iridium, gold, barium or bismuth salts, or the like. They can be triangular, for example, or they can be of other asymmetrical shape and further enhanced in their delineation of orientation by having multiple markers that change the projected pattern as a function of rotational orientation. The opening 1710, in an embodiment, further comprises one or more radiopaque marker 1714 denoting the extents of the opening 1710 to allow for positioning under fluoroscopy, further aided by the asymmetric marker 1712. The guidewire 402 is shown traversing the gap between the coupler 1706 and the expandable, releasable distal region 1702.

FIGS. 18A-C illustrate in more detail an expandable access sheath according to one embodiment of the invention. Additional details and further embodiments can be found in U.S. patent application Ser. No. 11/199,566, filed Aug. 8, 2005, the entirety of which is hereby incorporated by reference herein. FIG. 18A illustrates a radially expandable sheath 1800, wherein the sheath 1800 is in its collapsed, small diameter configuration. The sheath 1800 is configured for use in the gastrointestinal tract of the human or other animal. The proximal end of the sheath 1800 comprises the inner dilator shaft 1818, the outer dilator shaft 1824, and the dilator hub 1816. The dilator hub 1816 is integrally molded with, welded to, or is bonded thereto, to the guidewire port 1832. The dilator, or catheter, hub 1816 allows for gripping the dilator and it allows for expansion of the dilatation balloon 1820 by pressurizing an annulus between the inner dilator shaft 1818 and the outer dilator shaft 1824, said annulus having openings into the interior of the balloon 1820. The balloon 1820 is bonded, at its distal end, either adhesively or by fusion, using heat or ultrasonics, to the inner dilator shaft 1818. The proximal end of the balloon 1820 is bonded or welded to the outer dilator shaft 1824. In another embodiment, pressurization of the balloon 1820 can be accomplished by injecting fluid, under pressure, into a separate lumen in the inner or outer catheter shafts 1818 or 1824, respectively, said lumen being operably connected to the interior of the balloon 1820 by openings or scythes in the dilator tubing. Such construction can be created by extruding a multi-lumen tube, rather than by nesting multiple concentric tubes. The distal end 1804 generally comprises the distal sheath tube 1822 which is folded into one or more creases 1828 running along the longitudinal axis and which permit the area so folded to be smaller in diameter than the sheath tube 1806. The inner dilator shaft 1818 comprises a guidewire lumen 1834 that may be accessed from the proximal end of the dilator hub 1816 and passes completely through to the distal tip of the dilator shaft 1818. The guidewire lumen 1834 is able to slidably receive guidewires up to and including 0.038-inch diameter devices. The distal sheath tube 1804, in its collapsed configuration, can accept a removable shroud (not shown) that protects the distal sheath tube 1804 during shipping and handling and helps to maintain compression of the collapsed distal section 1804 prior to insertion in to the patient. The shroud is removed prior to inserting the sheath 1800 into a patient and will not pass over a guidewire without first removing the shroud to reveal the guidewire lumen 1834 on the dilator.

The distal end 1804 further comprises the dilator shaft 1818 and the dilatation balloon 1820. The dilator hub 1816 may removably lock onto the sheath hub 1808 to provide increased integrity to the system and maintain longitudinal relative position between the dilator shaft 1818 and the sheath tubing 1822 and 1806. The dilator hub 1816 is releasably affixed to the sheath hub 1808 by a snap, latch, bayonet mount, thread mount, or other quick-connect arrangement. The dilator hub 1816 is mated to the sheath hub 1808 so that it is held radially along its entire circumference or at a minimum of three points constraining against lateral relative axial movement in both directions orthogonal to the long axis of the sheath 1800. It is advantageous that the dilator hub 1816 be rotationally constrained within the sheath hub 1808 when they are mated so the operator cannot rotate the dilator hub and its attached balloon 1820 relative to the sheath hub 1808 and its attached distal sheath tube 1806. The dilator hub 1816 can be constrained to the sheath hub 1808 by a key arrangement with slots or dimples (not shown) in one component and protrusions (not shown) in the other component that are slidably received in the axial direction. When the sheath hub 1808 and the dilator hub 1816 are axially pulled apart, the rotational constraint is thereby disengaged.

The dilator shaft 1818 and the balloon 1820 are slidably received within the proximal sheath tube 1806. The dilator shaft 1818 and balloon 1820 are slidably received within the distal sheath tube 1822 when the distal sheath tube 1822 is radially expanded but are frictionally locked within the distal sheath tube 1822 when the tube 1822 is radially collapsed. The outside diameter of the distal sheath tube 1822 ranges from 4 French to 16 French in the radially collapsed configuration with a preferred size range of 5 French to 10 French. The outside diameter is critical for introduction of the device. Once expanded, the distal sheath tube 1822 has an inside diameter ranging from 8 French to 20 French. The inside diameter is more critical than the outside diameter once the device has been expanded. The wall thickness of the sheath tubes 306 and 322 ranges from 0.002 to 0.030 inches with a preferred thickness range of 0.005 to 0.020 inches.

FIG. 18B illustrates a cross-sectional view of the sheath 1800 of FIG. 18A wherein the balloon 1820 has been inflated causing the sheath tube 1822 at the distal end 1804 to expand and unfold the longitudinal creases or folds 1828. The distal sheath tube 1822 has the properties of being able to bend or yield, especially at crease lines, and maintain its configuration once the forces causing the bending or yielding are removed. The proximal sheath tube 1806 is affixed to the sheath hub 1808 by insert molding, bonding with adhesives, welding, or the like. The balloon 1820 has been inflated by pressurizing the annulus between the inner tubing 1818 and the outer tubing 1824 by application of an inflation device at the inflation port 1830 which is integral to, bonded to, or welded to the catheter hub 1816. The pressurization annulus is operably connect to the balloon 1820 at the distal end of the outer tubing 1824. Exemplary materials for use in fabrication of the distal sheath tube 1822 include, but are not limited to, polytetrafluoroethylene (PTFE), fluorinated ethylene polymer (FEP), polyethylene, polypropylene, polyethylene terephthalate (PET), and the like. A wall thickness of 0.008 to 0.012 inches is generally suitable for a device with a 16 French OD while a wall thickness of 0.019 inches is appropriate for a device in the range of 36 French OD. The resulting through lumen of the sheath 1800 is generally constant in French size going from the proximal end 1802 to the distal end 1804. The balloon 1820 is fabricated by techniques such as stretch blow molding from materials such as polyester, polyamide, irradiated polyethylene, and the like. In other embodiments, the inner lumen of the sheath 1800 within the distal end 1804 is greater than or less than the inner lumen of the sheath 1800 at the proximal end 1802.

FIG. 18C illustrates a side view of the sheath 1800 of FIG. 18B wherein the dilator shaft 1818, the balloon 1820, and the dilator hub 1816 have been withdrawn and removed leaving the proximal end 1802 and the distal end 1804 with a large central lumen capable of holding instrumentation. The shape of the distal sheath tube 1822 may not be entirely circular in cross-section, following expansion, but it is capable of carrying instrumentation the same size as the round proximal tube 1806. Because it is somewhat flexible and further is able to deform circumferentially, the sheath 1800 can hold noncircular objects where one dimension is even larger than the round inner diameter of the sheath 1800. The balloon 1820 is preferably deflated prior to removing the dilator shaft 1818, balloon 1820 and the dilator hub 1816 from the sheath 1800. The transition zone 1836 is shown in an exemplary embodiment wherein the proximal sheath tube 1806 is feathered into the distal sheath tube 1804 to provide a smooth transition in properties. The edges of the tubing at the transition zone 1836 appear, in an embodiment, as serrations. The serrations are preferably triangular in shape and between 0.1 and 5 cm long. The number of serrations can range between 1 and 20.

Referring to FIGS. 18A and 18B, the distal tubing 1804 further may comprise longitudinal runners or flutes separated by longitudinal slots or depressions. The folded distal sheath tube 1804 is constructed from materials that are plastically deformable, or malleable, such that the circumference is irreversibly increased by expansion of the dilator balloon 1820 and the outward forces created thereby. The wall thickness of the folded sheath tube 1804 is generally constant as the folded sheath tube 1804 is dilated. The folded sheath tube 1804, once dilated, will generally provide sufficient hoop strength against collapse that it keeps surrounding tissues open. The optional longitudinal runners or flutes separated by the slits or depressions provide a reduced friction track for the passage of instrumentation within the folded sheath tube 1804. The runners or flutes can be fabricated from materials such as, but not limited to, PTFE, FEP, PET, stainless steel, cobalt nickel alloys, nitinol, titanium, polyamide, polyethylene, polypropylene, and the like. The runners or flutes may further provide column strength against collapse or buckling of the folded sheath tube 1804 when materials such as calcific or cholesterol-based stones or other debris is withdrawn proximally through the sheath 1800. The runners or flutes may be free and unattached, they may be integral to the ID material, or they may be affixed to the interior of the folded sheath tube 1804 using adhesives, welding, or the like. In the case of flutes, the structure can be integrally formed with the folded sheath tube 1804, such forming generally occurring at the time of extrusion or performed later as a secondary operation. Such secondary operation may include compressing the folded sheath tube 1804 over a fluted mandrel under heat and pressure. The flutes may advantageously extend not only in the distal region 1804 but also in the interior of the proximal part of the sheath tubing 1806, and/or, but not necessarily the hub 1808.

The guidewire port 1832 is generally configured as a Luer lock connector or other threaded or bayonet mount. The guidewire is inserted therethrough into the guidewire lumen 1834 of the dilator tubing 1818 to which the guidewire port 1832 is operably connected. The guidewire port 1832 is preferably integrally fabricated with the dilator hub 1816 but may be a separately fabricated item that is affixed to the dilator hub 1816. A Tuohy Borst or other valved fitting is easily attached to such connectors to provide for protection against loss of fluids, even when the guidewire is inserted.

Referring to FIG. 18C, the proximal sheath tube 1806 further comprises a proximal reinforcing layer, an inner layer and an outer layer. The distal sheath tube 1804 further comprises a longitudinal fold 1828, a distal reinforcing layer, an outer layer, and an inner layer. The proximal reinforcing layer is embedded within the proximal sheath tube 1806, which is a composite structure, preferably formed from an inner and outer layer. The proximal reinforcing layer can be a coil, braid, or other structure that provides hoop strength and pushability to the proximal sheath tube 1806. The proximal reinforcing layer can be fabricated from metals such as, but not limited to, stainless steel, titanium, nitinol, cobalt nickel alloys, gold, tantalum, platinum, platinum iridium, and the like. The proximal reinforcing layer can also be fabricated from polymers such as, but not limited to, polyamide, polyester, and the like. Exemplary polymers include polyethylene naphthalate, polyethylene terephthalate, Kevlar, and the like. The proximal reinforcing layer, if it comprises metal, preferably uses metal that has been spring hardened and has a spring temper.

Further referring to FIG. 18C, the distal sheath tube 1804 is constructed from a composite construction similar to that of the proximal sheath tube 1806. The distal reinforcing structure, however, is not elastomeric but is malleable. The distal reinforcing structure is preferably a coil of flat wire embedded between the inner layer and the outer layer. The crease or fold 1828, shown in FIG. 18A, runs longitudinally the length of the distal sheath tube 1804 and is the structure that permits the distal sheath tube 18O4 to be compacted to a smaller diameter than its fully expanded configuration. There may be one fold 1828, or a plurality of folds 1828. The number of folds 1828 can range between 1 and 20, and preferably between 1 and 8, with the sheath tubing 1804 bendability and diameter having an influence on the optimal number of folds 1828.

The construction of the distal sheath tube 1804 can comprise a coil of wire with a wire diameter of 0.001 to 0.040 inches in diameter and preferably between 0.002 and 0.010 inches in diameter. The coil can also use a flat wire that is 0.001 to 0.010 inches in one dimension and 0.004 to 0.040 inches in the other dimension. Preferably, the flat wire is 0.001 to 0.005 inches in the small dimension, generally oriented in the radial direction of the coil, and 0.005 to 0.020 inches in width, oriented perpendicular to the radial direction of the coil. The outer layer has a wall thickness of 0.001 to 0.020 inches and the inner layer has a wall thickness of between 0.001 and 0.010 inches. The wire used to fabricate the coil can be fabricated from annealed materials such as, but not limited to, gold, stainless steel, titanium, tantalum, nickel-titanium alloy, cobalt nickel alloy, and the like. The wire is preferably fully annealed. The wires can also comprise polymers or non-metallic materials such as, but not limited to, PET, PEN, polyamide, polycarbonate, glass-filled polycarbonate, carbon fibers, or the like. The wires of the coil reinforcement can be advantageously coated with materials that have increased radiopacity to allow for improved visibility under fluoroscopy or X-ray visualization. The radiopaque coatings for the coil reinforcement may comprise gold, platinum, tantalum, platinum iridium, and the like. The mechanical properties of the coil are such that it is able to control the configuration of the fused inner layer and the outer layer. When the reinforcing layer is folded to form a small diameter, the polymeric layers, which can have some memory, do not generate significant or substantial springback. The sheath wall is preferably thin so that it any forces it imparts to the tubular structure are exceeded by those forces exerted by the malleable distal reinforcing layer. Thus, a peel away or protective sleeve is useful but not necessary to maintain the collapsed sheath configuration.

The inner layer and the outer layer preferably comprise some elasticity or malleability to maximize flexibility by stretching between the coil segments. Note that the pitch of the winding in the distal reinforcing layer does not have to be the same as that for the winding in the proximal reinforcing layer because they have different functionality in the sheath 1800.

Referring to FIGS. 18A, 18B, and 18C, due to stress hardening of the reinforcing layer and residual stress in the folded inner layer and outer layer, some remnant of the fold 1828 may still exist in the distal tube 1804. The expansion of the sheath 1800 in this configuration can be accomplished using a balloon 1820 with an internal pressure ranging between 3 atmospheres and 25 atmospheres. Not only does the balloon 1820 need to impart forces to expand the distal sheath tube 1804 against the strength of the reinforcing layer but it also needs to overcome any inward radially directed forces created by the surrounding tissue. In an exemplary configuration, a sheath 1800 uses a flat wire coil-reinforcing layer fabricated from fully annealed stainless steel 304V and having dimensions of 0.0025 inches by 0.010 inches. The coil has a pitch of 0.024 inches and allows the sheath to fully expand, at a 37-degree Centigrade body temperature, to a diameter of 16 French with between 4 and 7 atmospheres pressurization. The inner layer is polyethylene with a wall thickness of 0.003 to 0.005 inches and the outer layer is polyethylene with a wall thickness of 0.005 to 0.008 inches. The sheath 1800 is now able to form a path of substantially uniform internal size all the way from the proximal end to the distal end and to the exterior environment of the sheath at both ends. Through this path, instrumentation may be passed, material withdrawn from a patient, or both. A sheath of this construction is capable of bending through an inside radius of 1.5 cm or smaller without kinking or becoming substantially oval in cross-section.

The distal edge of the distal part of the sheath 1800 can comprise a fairing to smooth the transition between the small diameter dilator balloon 1820 of FIG. 18A and the folded sheath tubing 1804. The transition at the distal end of the folded sheath tubing 1804 can be sharp and require a fairing, which can be a cone of material, elastomeric or rigid, or it can be a bolus of material under the balloon 1820.

The expandable sheath 1800 can be fabricated in a small size and could include an integral (or separately introduced) small endoscope with a diameter of 1 to 2 mm with preferably forward-viewing capability and associated illumination channels operably connected to a light source operably connected to the proximal end of the endoscope. Such a combination could be maneuvered through the esophagus, stomach and duodenum. Optional steerable componentry including a flexion point proximal to the distal end of the sheath and pull wires and deflection mechanisms can facilitate the procedure. The sheath can be stabilized by a collar or balloon device so the forward looking scope could be stabilized and directed to access the sphincter either directly or with guidewire control. This would allow the endoscope operator to evaluate the nature of a stricture, for example, a stone blocking a duct could be assessed for size and position. Current use of fluoroscopy only denies the operator this visual assessment. Similarly, in the case of strictures, tissue could be assessed for pathology and visually directed biopsy could be accomplished by directly selecting the site of tissue sampling, with fluoroscopic guidance as an adjunctive, rather than a primary guiding methodology. Current methods of biopsy sampling are only 40% to 50% effective and this efficacy rate could be improved with the invention. Such an access system could incorporate sphincterotomy and balloon dilatation to permit the sheath to pass beyond obstacles.

The sheath can comprise an inflatable balloon to stabilize a small endoscope in a small sheath. The scope and/or sheath can accommodate a 0.035-inch, or larger, diameter guidewire through one of its lumens. The instrument channel or lumen in the endoscope can also accommodate baskets, graspers, or balloons, all of which can be operated within the view of the endoscope. A major consequence of pursuing gastrointestinal endoscopic diagnosis and therapy in this manner is the elimination of a 15 to 20 mm diameter endoscope to access, position, and visualize the duodenal wall to a point where the ampulla of Vater, the sphincter of Oddi, etc. can be identified. Once so positioned, much smaller devices are maneuvered through the sphincter of Oddi by scope rotation, followed by lateral deflection of guidewires and catheters followed by advancement through the sphincter. The patient is heavily sedated during this time to permit the unnatural esophageal occlusion that occurs during scope placement. The majority of cardiopulmonary complications occur as a result of the sedation required to accommodate the large scope passage and not the therapeutic gastrointestinal procedure itself.

Referring to FIGS. 18A and FIG. 2, in another embodiment, the sheath 1800 comprises an implant (not shown), which is detached and left within the sphincter of Oddi 206, said implant being either a one-way valve or a plug. The implant is beneficial because a surgical procedure of endoscopic origin dilates or cuts the sphincter of Oddi such that it, in some cases, no longer serves to prevent retrograde flow into the common bile duct 204 or the pancreatic duct 208. Dilation, or overdilation, can cause the sphincter of Oddi 206 muscle to become dysfunctional, or temporarily incontinent, for a short period of time such as one or more days, sufficient to cause pancreatitis and other complications. The plug, in an embodiment, can be fabricated from resorbable materials such as polylactic acid, polyglycolic acid, or other sugar or carbohydrate that ultimately dissolves. The valve can be a simple duck-bill valve that permits flow from the common bile duct 204 and pancreatic duct 208 into the descending duodenum 112. The valve can be. fabricated from silicone elastomer, C-Flex, or the like and have a seat that is fabricated from bioresorbable materials similar to those specified for the plug. The seat of the valve will dissolve over time and cause the valve to dislodge into the duodenum 112 from which it will eventually pass along with other fecal material. The valve seat or the plug can have antibiotics or other pharmacologic agents embedded or formed therein to minimize the chance of infection, for example. These agents can be encapsulated within microcapsules or microspheres to permit release over time or after a specified period of time. In another embodiment, the valve or plug are affixed to the exterior of the sheath 1800 so that when the sheath 1800 is removed, the valve or plug remain behind within the sphincter of Oddi 206.

The present invention may be embodied in other specific forms without departing from its spirit or essential characteristics. For example, the sheath may include instruments affixed integrally to the interior central lumen of the mesh, rather than being separately inserted, for performing therapeutic or diagnostic functions. The hub may comprise tie downs or configuration changes to permit attaching the hub to the mouth, nose, or face of the patient. The dilatation means may be a balloon dilator as described in detail herein, it may rely on axial compression of a braid to expand its diameter, or it may be a translation dilator wherein an inner tube is advanced longitudinally to expand an elastomeric small diameter tube. Dilation may also occur as a result of unfurling a thin-film wrapped tube or by rotation of a series of hoops so that their alignment is at right angles to the long axis of the sheath. The embodiments described herein further are suitable for fabricating very small diameter catheters, microcatheters, or sheaths suitable for cardiovascular or neurovascular access. These devices may have collapsed diameters less than 3 French (1 mm) and expanded diameters of 4 to 8 French. Larger devices with collapsed diameters of 16 French and expanded diameters of 60 French or larger are also possible. Such large devices may have airway or lower gastrointestinal tract applications, for example, the latter being accessed via laparoscopy, oral, or a rectal approach, for example. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

What is claimed is:

1. An expandable transluminal access sheath for providing minimally invasive access to a gastrointestinal tract, comprising:
an axially elongate sheath tube comprising a proximal end, a distal end, and a through lumen extending therethrough, the sheath tube further comprising a distal region that is expandable from a collapsed configuration to an expanded configuration in response to outward pressure applied therein;
a hub coupled to the proximal end of the sheath tube; and
an obturator extending through the hub and sheath tube, the obturator configured to occlude the central lumen of the sheath tube during insertion of the sheath tube into the gastrointestinal tract, the obturator comprising an obturator hub that is releasably coupled to the hub of the sheath and a guidewire lumen that extends through the obturator, the obturator further comprising a balloon dilator capable of expanding the distal region of the sheath from the collapsed configuration to the expanded configuration.

2. The transluminal sheath of claim 1 where the sheath tube comprises a reinforcing layer embedded within a membrane layer comprising a polymeric material.

3. The transluminal sheath of claim 1 wherein the sheath tube comprises: an outer layer, an inner layer, and a reinforcing layer, the outer layer and the inner layer comprising polymeric materials.

4. The transluminal sheath of claim 3 wherein the reinforcing layer is a coil of metal.

5. The transluminal sheath of claim 3 wherein the reinforcing layer is a braid.

6. The transluminal sheath of claim 3 wherein the inner and outer layer are fabricated from different polymeric materials.

7. The transluminal sheath of claim 1 wherein the length of the sheath tube is between about 150 and about 250 cm.

8. The transluminal sheath of claim 1, wherein the through lumen of the sheath tube has a diameter between about 6 and about 20 French when the distal region is the expanded configuration.

9. A method of instrumenting a body lumen comprising the steps of:
inserting an endoscope with a working channel into a patient;
positioning an exit point of the working channel beside an entrance to a branch of the body lumen,
routing a guidewire down the working channel of the endoscope and into the branch of the body;
positioning an end of the guidewire at a target location within the body lumen;
removing the endoscope from the patient leaving the guidewire in place,
inserting a sheath with a collapsed distal region and a pre-inserted dilator into the patient over the guidewire;
advancing the sheath to a treatment site within the side branch of the body lumen;
dilating the distal region of the sheath so that the distal region of the sheath is expanded;
collapsing the dilator;
removing the dilator from the sheath,
inserting instrumentation through the lumen of the sheath,
performing therapy or diagnosis with the instrumentation, and
removing the sheath from the patient.

10. The method of claim 9 wherein dilating the distal region comprises inflating a balloon on the dilator.

11. The method of claim 9 wherein dilating the distal region comprises attaching a liquid-filled inflation device to a balloon inflation port at proximal end of the dilator and infusing liquid under pressure into the dilator.

12. The method of claim 11 wherein collapsing the dilator comprises withdrawing a plunger on an inflation device to withdraw liquid from the dilator.

13. The method of claim 9 wherein dilating of the sheath comprises dilating a sphincter surrounding at least a portion of an expandable region of the sheath.

14. The method of claim 9 wherein performing therapy or diagnosis comprises removing stones from the branch.

15. The method of claim 14 wherein the stones are removed with graspers and are pulled to a window in the sheath.

16. The method of claim 9 wherein the lumen of the expanded distal region of the sheath is substantially larger than the lumen of the proximal non-expandable region.

17. The method of claim 9 wherein the lumen of the expanded distal region is substantially smaller than that of the lumen of the proximal non-expanded region.

18. The method of claim 9 wherein the expanded lumen created in the expandable region by the dilator is substantially the same size as that of the proximal sheath lumen.

19. The method of claim 9 further comprising the step of separating the expandable region from the non-expandable region by selective actuation of a coupler release mechanism prior to removal of the non-expandable region from the body.

20. A access device for insertion into a gastrointestinal tract, comprising:
means for tracking over a guidewire to a target treatment site;
means for diametrically collapsing at least a distal end of the sheath;
means for dilating at least a portion of the distal end of the sheath, from the proximal end of the sheath, and
means for removal of the sheath from the patient body lumen or cavity.

21. The sheath of claim 20 further comprising means for performing instrumentation, infusion of material into the body lumen or cavity, or withdrawal of material from the body lumen or cavity.

22. The sheath of claim 20 further comprising means for maintaining an open lumen in the small body lumen following removal of at least a portion of said sheath.

23. The sheath of claim 20 further comprising means for readily visualizing, positioning, and orienting said sheath using visualization techniques employing X-rays.

24. An expandable transluminal access sheath adapted for providing minimally invasive access to the gastrointestinal tract through a working channel of an endoscope, comprising:
an axially elongate sheath tube with a proximal end, a distal end, and a central through lumen;
a distal region of the sheath which is expandable, in response to outward pressure applied therein, to a diameter which is larger than that of a proximal region of the sheath,
a hub affixed to the proximal end of the sheath tube, the hub adapted to facilitate the passage of instrumentation;

25. The transluminal sheath of claim 24, comprising:
an obturator, which serves to occlude the central lumen of the sheath during insertion, the obturator comprising a hub that releasably locks to the hub of the sheath; and
a guidewire lumen within the obturator, capable of passing over standard medical guidewires and which will allow the obturator and sheath to track over said guidewires;
wherein the obturator is a balloon dilator capable of expanding the distal region of the sheath from a collapsed configuration to an expanded configuration.

26. The transluminal sheath of claim 24 wherein the distal expandable region comprises an opening.

27. The transluminal sheath of claim 24 further comprising a releasable coupler which reversibly couples the expandable distal region of the sheath to a proximal region of the sheath.

28. The transluminal sheath of claim 24 further comprising a window opening which can be aligned with a branch lumen to permit flow from that branch lumen into the sheath, along with flow from a main lumen.

29. The transluminal sheath of claim 28 further comprising radiopaque markings that are asymmetric and capable of providing rotational position information when their image or shadow is projected onto a two-dimensional plane.

30. The transluminal sheath of claim 28 further comprising radiopaque markers that denote the location and extents of the window.
